(19) United States
(12) Patent Application Publication
(10) Pub. No.: US 2012/0108909 A1
(43) Pub. Date: May 3, 2012

(54) Assessment and Rehabilitation of Cognitive and Motor Functions Using Virtual Reality

(75) Inventors: Semyon Slobounov, Chicago, IL (US); Elena Slobounov, Chicago, IL (US)

(73) Assignee: HeadRehab, LLC, Chicago, IL (US)

(21) Appl. No.: 12/938,551

(22) Filed: Nov. 3, 2010

(51) Int. Cl. A61B 5/00

(52) U.S. Cl. 600/300

(57) ABSTRACT

A user-friendly reliable process is provided to help diagnose (assess) and treat (rehabilitate) impairment or deficiencies in a person (subject or patient) caused by a traumatic brain injury (TBI) or other neurocognitive disorders. The economical, safe, effective process can include: generating and electronically displaying a virtual reality environment (VRE) with moveable images; identifying and letting the TBI person perform a task in the VRE; electronically inputting and recording the performance data with an electronic interactive communications device; electronically evaluating the person's performance and assessing the person's impairment by electronically determining a deficiency in the person's cognitive function (e.g. memory, recall, recognition, attention, spatial awareness) and/or motor function (i.e. motor skills, e.g. balance) as a result of the TBI or other neurocognitive disorder.

### BACKGROUND OF THE INVENTION

This invention relates to traumatic brain injuries, (including mild traumatic brain brain injuries, known as ‘concussion’), and more particularly, to assessing and treating impairment caused by traumatic brain injuries (TBI). Further applications for this invention have been discovered in various areas of neurological abnormality and neurocognitive deficiency.

Currently there is no “golden standard” for assessment and rehabilitation of the neurocognitive effects of concussion. Over the years, various techniques and procedures have been developed or suggested to treat traumatic brain injuries (TBI). These prior techniques and procedures, although controversial, have met with varying degrees of success.

In the past as well as in a current conventional clinical practice, initial neurological examination of patients older than 4 years has included evaluation using the Glasgow Coma Scale (GCS), which assigns points for eye opening, verbal response and motor response. A score of 13 to 15 indicates a mild TBI, a score of 9 to 12 indicates moderate TBI, and a score of 8 or lower indicates severe TBI. In a civilian hospital in the United States, 80% of patients admitted for TBI will have a score in the mild range. Diagnostic imaging is used in determining the location and extent of brain trauma and is helpful in determining possible sequelae.

Recent research has shown many shortcomings of current TBI assessments rating scales, neuropsychological assessments and brain imaging techniques (CT, conventional MRI, fMRI, DTI, MRS & EEG) in showing accurate impact of TBI in the sub-acute phase of injury (beyond 7 days post injury) and in the chronic phase of injury (over 30 days post injury). As a result, severity of TBI remains unclear because objective anatomic pathology is rare and the interaction among cognitive, behavioral and emotional factors can produce enormous subjective symptoms in an unspecified manner.

The conventional ImPact technique has been a popular test in the market that administers pre- and post-injury testing using computerized versions of paper and pencil tests. The ImPact technique is focused mainly on assessment of cognitive and executive functions (e.g., memory and attention) but does not address other serious effects of TBI, such as balance. Listed hereinafter are the components of the ImPact test:

| Test Name | Neurocognitive Domain Measured |
|---|---|
| Word Memory | Verbal recognition memory (learning and retention) |
| Design Memory | Spatial recognition memory (learning and retention) |
| X's and O's | Visual working memory and cognitive speed |
| Symbol Match | Memory and visual-motor speed |
| Color Match | Impulse inhibition and visual-motor speed |
| Three letter memory | Verbal working memory and cognitive speed |
| Symptom Scale | Rating of individual self-reported symptoms |

Both traditional pen-and-pencil procedures and the ImPact computerized testing summarized above may provide the ability to initially assess and to track the recovery following TBI but do not focus on long term assessment and offer limited or no rehabilitative options.

There are, however, a few factors that may potentially bias the results of multiple neuropsychological tests, mainly the practice effect, subjects' efforts and their motivation. Furthermore, the majority of clinically-based assessments of cognitive functioning has internal validity, but can lack both external and ecological validity.

In the military, 27% of patients suffering from primary blast-related TBI treated at the Brain Injury Center—Vestibular & Balance experienced balance problems up to 13-18 weeks post-injury. Moreover, there are numerous cases when visually-induced postural problems and disorientation persist up to one year post-injury in war fighters who were returned to active duties. The previously mentioned conventional tests do not address these longer-term problems.

Also, the problem with an accurate assessment of TBI's impact, especially using the current Military Acute Concussion Evaluation (MACE), is exaggerated by recent concern raised by doctors at the Defense and Veteran Brian Injury Center (DVBIC). The MACE oral evaluation of service members in combat has been used since 2000 at the scene of an accident or bombing or shortly after an incident occurred in the field. According to a report by Catherine Donaldson-Evens, “Brian-Injury tests changed because troops were caught cheating.” (FOXNEWS.COM, Nov. 19, 2007) Soldiers had been supplying each other with answers to prior exams so they can pass and remain with their units on the battlefield. Clearly, the possibility of cheating during concussion subjective testing may put servicemen and women at high risk for recurrent concussions as well as putting their comrades in extreme danger. Therefore, needs for technologically advanced assessment tools, eliminating the possibility of cheating and/or misinterpretation are obvious.

Other prior tools and conventional techniques available in the market that utilize virtual reality (VR) as a tool for neurological and cognitive work are the IREX and VR Psychology techniques. The conventional IREX technique is focused on physical therapy using the virtual reality environment to rehabilitate patients with limited mobility. Some of the work may impact the balance or have an impact on the memory through repetition of the physical exercises; however, the conventional IREX system does not focus on TBI and does not directly track the neurocognitive improvements outside of the physical recovery.

The conventional VR Psychology technique has been popular to treat Post Traumatic Stress Disorder (PTSD). The military facilities utilize the conventional VR Psychology technique to re-create the locations of deployment where the soldiers have suffered from a blast related injury caused by a car bomb or other form of explosion (for example, see ‘Virtual Iraq’). The conventional VR Psychology technique sometimes allows the patient to overcome the fear associated with the incident in a safe environment.

In summary, the conventional ImPact and other conventional techniques have limited neurocognitive assessment ability, particularly with long term (LT) effects, a lack of comprehensive rehabilitation and LT impact rehabilitation, limited neurocognitive baseline, are not cheating-proof, have a strictly TBI focus and are not transferable to real-life. They may be reimbursable by insurance but only require computer hardware and software and do not leverage virtual reality to produce highly accurate responses in subjects.

The conventional IREX technique also has limited feasibility and accuracy of neurocognitive assessment, limited LT impact assessment, limited comprehensive rehabilitation and LT impact rehabilitation, no neurocognitive baseline and does not focus on TBI. While IREX is transferable to real-life situations, is reimbursable by insurance and has a hardware and software system, it does not operate on a virtual reality platform.

The conventional VR Psychology technique further has limited neurocognitive assessment, no LT impact assessment, limited comprehensive rehabilitation and LT impact rehabilitation, no neurocognitive baseline and a psychological rehabilitation focus for PTSD rather than TBI. The VR Psychology technique is used as a gaming tool rather than comprehensive TBI data gathering system, can be transferable to real-life situations, is reimbursable by insurance and has a hardware and software system and VR platform.

The conventional MACE technique has limited neurocognitive assessment, no LT impact assessment, no comprehensive rehabilitation, no LT impact rehabilitation or neurocognitive baseline, nor is it cheating-proof nor free of learning from repeated testing. The conventional MACE technique can have TBI focus but is not transferable to real-life situations, is not reimbursable by insurance and has no hardware and software system or VR platform.

Diagnosing and treating TBI caused by football, soccer, rugby, baseball and other contact sports injuries has become very important, as well as diagnosing and treating TBI caused by work related accidents, vehicle accidents, stairway accidents and other accidents occurring at home, in addition to incidents at the military base. Unfortunately, many of the conventional techniques and procedures for diagnosing and treating TBI are ineffective, limited, expensive, burdensome, cumbersome, and unreliable and lack accuracy in long term assessment and rehabilitation.

In addition to TBI, several other areas of neurocognitive deficiencies lack comprehensive baseline, assessment and rehabilitation tools. These deficiencies include but are not limited to Parkinson's Disease, Alzheimer's Disease, Pediatric Concussions, ADHD, elderly care and patients post-stroke.

It is, therefore, desirable to provide an improved process for use with a person having a traumatic brain injury (TBI) or other neurocognitive deficiency, which overcomes most, if not all, of the preceding problems with existing systems. It should be noted that most existing techniques for assessment of neurocognitive and behavioral deficits are not challenging enough to observe residual long-term neurocognitive abnormalities especially in the sub-acute phase of injury. Injured subjects may use various compensatory strategies to successfully accomplish these other testing protocols and appear to be asymptomatic. Those residual long-term deficits can be detected if more challenging and demanding testing procedures are implemented. More severe concussions are sensitive to a changing degree of complexity in the tasks. The applications proposed in this invention directly address this important clinical pursuit by offering a widely variable degree of challenge for the subject via the virtual reality platform. As the clinician alters this degree of challenge using the virtual environment, the subject must use different amounts of effort to complete the task at hand. Other tests make it difficult to assess the amount of effort a patient must put into the test. The proposed applications allow the clinician to increase the challenge complexity to find the subject's maximum range of effort.

### BRIEF SUMMARY OF THE INVENTION

An improved process is provided for use with a person (subject or patient) having a traumatic brain injury (TBI) or other neurocognitive deficiency. The reliable process is especially helpful to diagnose (assess) and treat (rehabilitate) impairment or deficiencies caused by traumatic brain injuries and other neurological disorders. Advantageously, the novel process is safe, comprehensive, accurate, easy to use, effective and efficient. Desirably, the user-friendly process is economical, comforting to the patient and very helpful to medical personnel. The TBI diagnostic (assessment) process has demonstrated accuracy and higher sensitivity in greater areas of neurocognitive testing over longer periods of time post-injury.

The novel process can include: selecting a test area from the main menu; generating a virtual reality environment (VRE) comprising at least one image with a central processing unit; electronically displaying the VRE to a person (subject or patient) having a TBI or other neurological deficiency; identifying specific tasks to be performed by the person in the VRE; performing the task by the person; electronically inputting results from the interaction comprising the performance and responses of the person to the CPU through an electronic interactive device; electronically evaluating the person's performance in the CPU based on the electronically inputted interactive communications to the CPU; and electronically assessing the person's specific and overall impairment scores by electronically processing individual deficiencies in the person's cognitive function (e.g. memory, recall, recognition, attention, spatial awareness) and motor function (i.e. motor skills, e.g. balance) as a result of the TBI.

The task can comprise, but is not limited to: object recognition (e.g. recognition of virtual objects), virtual navigation, virtual walking, virtual steering, spatial navigation, object navigation spatial memory, kinesthetic imagery, virtual arrangement of images, standing, balancing and memorizing virtual objects. The VRE can be a three-dimensional (3D) virtual reality environment, the image can be moveable and the task can be performed by the person with the aid of interactive communications device(s).

The image can comprise a virtual image of one or more of the following, but is not limited to: an elevator, hospital corridor, body, room, pathway, object, hospital room, bathroom, door, hall, wall, sign, picture, bed, floor, cart, person, table, positions of a person's body, and furniture. The VRE can be accompanied by one or more visual distractions and/or audible distractions.

The performance data can be electronically inputted and recorded in the CPU and electronically reported (e.g. electronically outputted, e-mailed, transmitted or printed) from the CPU. The performance data can be electronically compared with the person's prior performance data or normal performance data from a data base. The performance data and comparison data can be electronically scored. The score and comparison data can be electronically reported from the CPU and used to help rehabilitate the person (subject or patient) having and suffering from a TBI or other neurological deficiency.

The inventive virtual reality (VR) system and process are designed to assess and rehabilitate cognitive abnormalities in subjects with traumatic brain injury (TBI), including mild TBI (which is also know as a concussion), or other conditions that impact brain function. The novel system and process can be used within athletic organizations, research institutes, hospitals and the military community to gather normative and post-injury data on athletes, patients, test subjects and soldiers. From this data, the novel system and process can compile an assessment of the subject's current brain function that can be compared to normative data or past test data for that subject.

Post assessment, the user-friendly system and process can function as a technique and/or as a set of tools, for the rehabilitation of deficient areas of cognitive function and motor function. The user-friendly system and process can include software modules that focus on studying and addressing critical areas of brain function and primary memory (recall and recognition), attention, balance, visual-kinesthetic integration and spatial awareness. The assessment modules can use computer generated virtual reality environments to recreate everyday activities, such as walking or otherwise navigating down a hallway or reacting to movement within the virtual environment. The CPU and software can capture subject response data and measure the ability of the subject (patient) to perform various tasks. The CPU and software can then compile a quantitative assessment of the subject's experience within the virtual environment. The results of each subject can be scored individually and can also be compared to a normal baseline of healthy subjects as well as any previous data gathered on that specific subject. Clinicians and physicians can then make a final diagnosis of the subject's current cognitive function to determine any areas of deficiency and prescribe appropriate treatment. The clinicians can also utilize the rehabilitation software modules to treat the subject while regularly gauging the progress of the subject with the assessment software modules. The rehabilitation modules can be altered each time in order to avoid the learning impact and address the issue of differential responsiveness as a function of injury severity.

When compared to commonly used paper and pencil tests of cognitive function, the inventive system and process provides an advanced tool that can allow the subject, while under the supervision of a medical professional, to be immersed or placed in a controlled, non-invasive, realistic virtual reality environment. The subject's experience in the virtual environment of the inventive process and system is ecologically valid and offers natural neurocognitive and motor response as well as true transferability to real life situations. The inventive process and system can be offered on both stationary and portable VR platforms and can be adapted to the specific needs of the patient and medical professional. With further developments in hardware, the VR platforms may include head mounted displays, high-resolution VR goggles and 3D televisions.

The inventive system, process and software can use virtual reality to create ecologically valid, realistic virtual environments that are transferable to real life and create a sense of presence in the virtual environment for the subject. The inventive system, process and software can be used alone or in conjunction with brain imaging technology [i.e. functional magnetic resonance imaging (fMRI), electroencephalography (EEG)] to accurately assess multiple areas of cognitive motor function [i.e. attention, balance, recognition, spatial navigation, praxis] and rehabilitate areas of cognitive motor function found to be deficient. In addition, motion tracking devices [i.e. force platform, Vicon, accelerometers, etc.] can be used in conjunction with the software to provide additional data from the subject's virtual experience and responses to VR scene manipulations.

The novel process and system can successfully address the limitations of the techniques and tools currently used in the market and can offer a comprehensive technique and tool for the objective assessment of a patient's dysfunction and a customized rehabilitation program for the patient's individual deficiencies.

The assessment and rehabilitation modules of the novel process and system can provide both a technique to assess (diagnose) and rehabilitate (treat) cognitive functions and motor functions. The inventive system, process and software can provide assessment of multiple cognitive functions and motor functions, as well as help rehabilitate cognitive functions and motor functions found to be dysfunctional. The software assessment and rehabilitation modules can include cognitive and motor functions, such as, but not limited to, attention, balance, memory, visual kinesthetic integration function (body) and spatial awareness.

The virtual reality environment of the novel process and system can immerse or place the subject (patient) into a three-dimensional (3D) virtual environment to create a sense of presence and invoke a realistic response from the subject's neurocognitive system producing objective and productive results.

The novel process and system can provide subject interaction and data gathering, such as with interactive devices which allow interaction with software and the virtual environment and can send subject response data to the CPU, as well as with interactive devices that can be custom fit for a special-needs subject. The novel process and system can also provide: subject interaction and data gathering, such as by storing subject response data in the CPU throughout the testing period to be accessed with the reporting module for automatic subject classification and results calculation and display. The novel process and system can further provide subject interaction and data gathering, such as with computer-generated random tests which help prevent an undesirable learning effect and maintain objectivity of the testing process.

The inventive system, process and software can standardize quantitative results and scoring method to allow for relative comparison of subject scores as the injury evolved from acute to sub-acute to chronic phases.

The reporting modules of the novel process and system can specifically target data to be gathered from each test, classify the subject's performance, and report individual module test results as well as provide a comprehensive report for overall cognitive functions and motor functions of the subject (patient).

The novel process and system can also provide a relative results scoring system that generates and reports a relative, quantitative cognitive score (e.g. 6.5) on a proprietary, standardized scale, which allows for relative comparison of subjects results against a baseline of normative (normal) data over time and against other subjects, as well as well as against collected data from a database and easily identify and track rehabilitation progress via quantitative scoring.

The novel assessment process can measure the cognitive and motor effects of the injury in the acute phase, directly after impact and also be used repeatedly during the sub-acute and chronic phases. Repeated assessment can discern patterns of recovery, deterioration or unchanged states of the subject's neurocognitive function. Repeated assessment through the evolution of the injury over time could define the probability of long term deficit for various impacted cognitive and motor functions.

The inventive system, process and software can provide an accurate neurocognitive assessment, long term (LT) impact assessment, comprehensive rehabilitation, LT impact rehabilitation, a neurocognitive baseline, a hardware and software system and a virtual reality (VR) platform. The inventive system, process and software can also be cheating-proof and can further provide a focus on TBI or other neurocognitive dysfunction, as well as can be transferable to real-life situations and can be reimbursable by insurance.

A more detailed explanation of the invention is provided in the following detailed descriptions and appended claims taken in conjunction with the accompanying drawings.

### BRIEF DESCRIPTION OF THE DRAWINGS

FIG. 1 is a diagrammatic view of a central processing unit (CPU), interactive communication devices and related equipment for use with the traumatic brain injury (TBI) diagnostic process (assessment) and treatment process (rehabilitation) in accordance with principles of the present invention.

FIG. 2 is a master menu flowchart of the TBI diagnostic process (assessment) and treatment process (rehabilitation) in accordance with principles of the present invention.

FIG. 3 is a front view of a virtual hospital corridor with an object comprising a wheelchair for use with the TBI diagnostic process (assessment) and treatment process (rehabilitation) in accordance with principles of the present invention.

FIG. 4 is a back view of a person (subject) with a safety harness facing a virtual hospital corridor for use in the TBI diagnostic process (assessment) and treatment process (rehabilitation) in accordance with principles of the present invention.

FIG. 5 is a front view of a virtual hospital corridor for use with the TBI diagnostic process (assessment) and treatment process (rehabilitation) in accordance with principles of the present invention.

FIG. 6 is a back view of a person (subject) with a safety harness, standing on a force platform that is built into the floor and navigating a virtual hospital corridor for use in the TBI diagnostic process (assessment) and treatment process (rehabilitation) in accordance with principles of the present invention.

FIG. 7 is a top view of a virtual hospital corridor system for use with the TBI diagnostic process (assessment) and treatment process (rehabilitation) in accordance with principles of the present invention.

FIG. 8 is a front view of a virtual hospital corridor with an object comprising a walker for use with the TBI diagnostic process (assessment) and treatment process (rehabilitation) in accordance with principles of the present invention.

FIG. 9 is a front diagrammatic view of virtual reality objects for the person (subject) to memorize in the TBI diagnostic process (assessment) and treatment process (rehabilitation) in accordance with principles of the present invention.

FIG. 10 is an interactive panel with a front diagrammatic view of virtual reality objects for the person (subject) to select in the TBI diagnostic process (assessment) and treatment process (rehabilitation) in accordance with principles of the present invention.

FIG. 11 is a front view of a virtual hospital corridor with an object comprising a chair for use with the TBI diagnostic process (assessment) and treatment process (rehabilitation) in accordance with principles of the present invention.

FIG. 12 is a front view of a virtual hospital corridor with a red dome over a previously selected object comprising a chair for use with the TBI diagnostic process (assessment) and treatment process (rehabilitation) in accordance with principles of the present invention.

FIG. 13 is a back view of a person (subject) with a safety harness facing a virtual elevator for use in the TBI diagnostic process (assessment) and treatment process (rehabilitation) in accordance with principles of the present invention.

FIG. 14 is a front view of a virtual elevator with elevator buttons and the door open on the fifth floor for use with the TBI diagnostic process (assessment) and treatment process (rehabilitation) in accordance with principles of the present invention.

FIG. 15 is a back view of a person (subject) with a safety harness facing a virtual hospital room for use in the TBI diagnostic process (assessment) and treatment process (rehabilitation) in accordance with principles of the present invention.

FIG. 16 is a front view of a virtual hospital room for use with the TBI diagnostic process (assessment) and treatment process (rehabilitation) in accordance with principles of the present invention.

FIG. 17 is a back view of a person (subject) with a safety harness looking at virtual body positions for sitting and standing for use in the TBI diagnostic process (assessment) and treatment process (rehabilitation) in accordance with principles of the present invention.

FIG. 18 is a front view of virtual body positions for sitting and standing for use in the TBI diagnostic process (assessment) and treatment process (rehabilitation) in accordance with principles of the present invention.

FIG. 19 is a front view of the first page of a report which was outputted from a reporting module for use in the TBI diagnostic and rehabilitation process in accordance with principles of the present invention.

FIG. 20 is a front view of a virtual environmental screen snapshot showing an object (prop) comprising a couch and six walls comprising a ceiling, floor, front wall, back wall and left and right side walls, for use in the TBI diagnostic process (assessment) and treatment process (rehabilitation) in accordance with principles of the present invention.

FIG. 21 is a Spatial Memory 1 Module flowchart of the TBI diagnostic process (assessment) and treatment process (rehabilitation) in accordance with principles of the present invention.

FIG. 22 is a Spatial Memory 2 Module flowchart of the TBI diagnostic process (assessment) and treatment process (rehabilitation) in accordance with principles of the present invention.

FIG. 23 is a Memory 3A Module flowchart of the TBI diagnostic process (assessment) and treatment process (rehabilitation) in accordance with principles of the present invention.

FIG. 24 is a Memory 3B Module flowchart of the TBI diagnostic process (assessment) and treatment process (rehabilitation) in accordance with principles of the present invention.

FIG. 25 is an Attention Module flowchart of the TBI diagnostic process (assessment) and treatment process (rehabilitation) in accordance with principles of the present invention.

FIG. 26 is a Balance Module flowchart of the TBI diagnostic process (assessment) and treatment process (rehabilitation) in accordance with principles of the present invention.

FIG. 27 is a Body Awareness Module flowchart of the TBI diagnostic process (assessment) and treatment process (rehabilitation) in accordance with principles of the present invention.

FIG. 28 is a Reporting Module flowchart of the TBI diagnostic process (assessment) and treatment process (rehabilitation) in accordance with principles of the present invention.

FIG. 29 is a Build Virtual Environment (VE) flowchart for use in the TBI diagnostic process (assessment) and treatment process (rehabilitation) in accordance with principles of the present invention.

FIG. 30 is a Build Props flowchart for use in the TBI diagnostic process (assessment) and treatment process (rehabilitation) in accordance with principles of the present invention.

FIG. 31 is a Build VE Perturbation flowchart for use in the TBI diagnostic process (assessment) and treatment process (rehabilitation) in accordance with principles of the present invention.

FIG. 32 is a Build Path Segments flowchart for use in the TBI diagnostic process (assessment) and treatment process (rehabilitation) in accordance with principles of the present invention.

FIG. 33 is a Connect Interactive Devices flowchart for use in the TBI diagnostic process (assessment) and treatment process (rehabilitation) in accordance with principles of the present invention.

FIG. 34 is a Build Virtual Environment (VE) Interactive Panel flowchart for use in the TBI diagnostic process (assessment) and treatment process (rehabilitation) in accordance with principles of the present invention.

FIG. 35 is a flowchart diagram key for the TBI diagnostic process (assessment) and treatment process (rehabilitation) in accordance with principles of the present invention.

FIG. 36 is a chart of a truncated piece of code defining a block data structure for use in the TBI diagnostic process (assessment) and treatment process (rehabilitation) in accordance with principles of the present invention.

FIG. 37 is a diagram of a model representing a list or linked list of block data structures where the solid blocks represent a null pointer terminating the list for use in the TBI diagnostic process (assessment) and treatment process (rehabilitation) in accordance with principles of the present invention.

FIG. 38 is a diagram of a model representing a doubly linked list of six block data structures to hold the data for building a virtual environment with six blocks positioned in two rows and three columns for use in the TBI diagnostic process (assessment) and treatment process (rehabilitation) in accordance with principles of the present invention.

FIG. 39 is a diagram of a model representing a complex linked list of six block data structures with additional links providing fast access to an adjacent block in the next or previous column for use in the TBI diagnostic process (assessment) and treatment process (rehabilitation) in accordance with principles of the present invention.

### DETAILED DESCRIPTION OF THE INVENTION

The following is a detailed description and explanation of the preferred embodiments of the invention and best modes for practicing the invention.

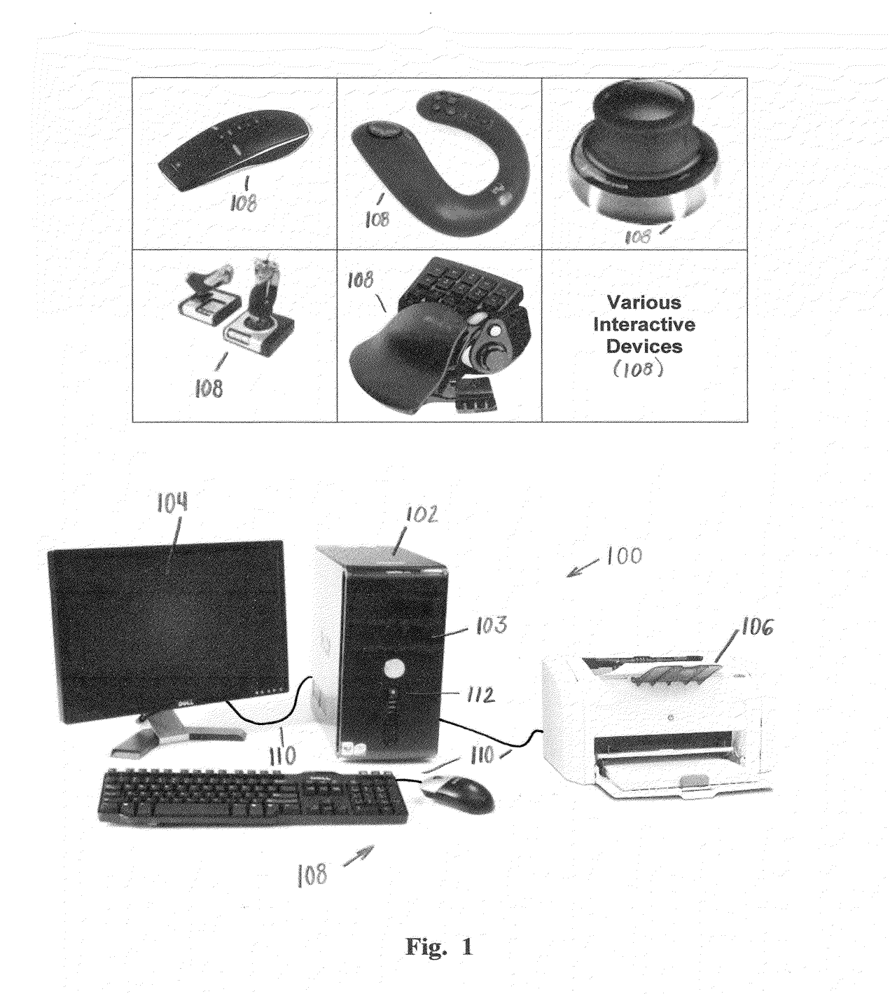

As shown in FIG. 1 of the drawings, a traumatic brain injury (TBI) diagnostic (assessment) and rehabilitative process and system 100 can have a central processing unit (CPU) 102 including a hard drive 103 which provides data storage. The CPU can have various related equipment and components including a screen 104, printer 106, and one or more interactive communications devices 108. The CPU can be hard wired by a bundle of wires or cable 110 and/or or in wireless communication, such as by Bluetooth, via an antenna 112 with one or more related equipment and components, e.g. the screen, printer, and interactive communications device. If desired, the screen can be separate from and/or operatively associated with the CPU.

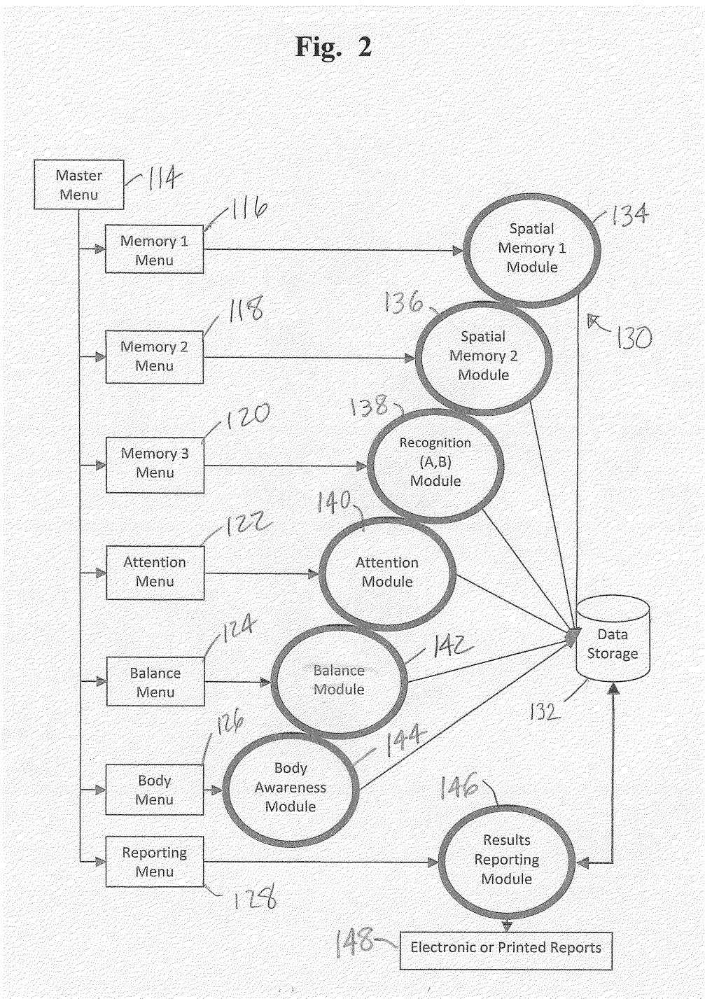

The master menu flowchart of FIG. 2 has a master menu 114 which can comprise a Memory 1 menu 116, a Memory 2 menu 118, a Memory 3 menu, 120, an Attention menu 122, a Balance menu 124, a Body menu 126, and a Reporting menu 128. The menus are provided for use with and input into the modules 130 which can transmit and send their electronic output and data to data storage 132, such as a hard drive, universal serial bus (USB) flash drive, computer disc or another CPU. The modules include: a Spatial Memory 1 Module 134 for use with and which inputs into the Memory 1 menu; a Spatial Memory 2 Module 136 for use with and which inputs into the Memory 2 menu; a Recognition (A, B) Module 138 for use with and which inputs into the Memory 3 menu; an Attention Module 140 for use with and which inputs the Attention menu; a Balance Module 142 for use with and which inputs the Balance menu; a Body Awareness Module 144 for use with and which input the Body menu; a results Reporting Module 146 for use with and which inputs the Reporting menu. The results Reporting Module can transmit and send its output to generate reports 148 such as electronic reports or printed reports via the printer. The results Reporting Module can also send and receive data from data storage.

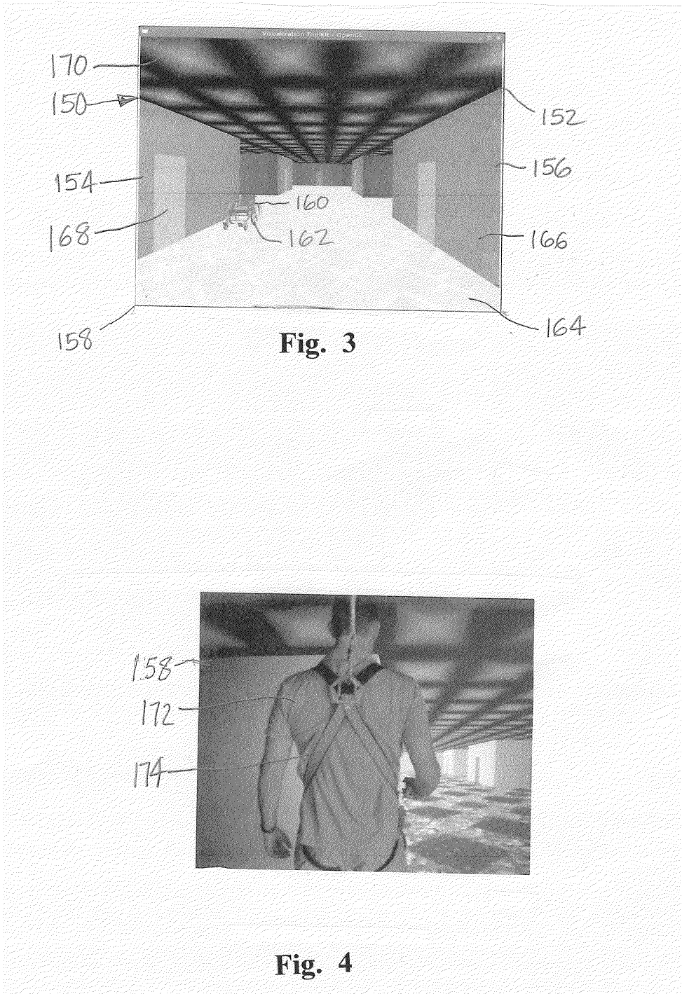

The CPU can generate a virtual reality environment (VRE) 150 (FIG. 3) providing a virtual environment (VE) with a virtual scene 152 and one or more virtual images 154, preferably moveable three-dimensional (3D) images 156. The VRE can be generated by the CPU with the modules and menus. In FIG. 3 and FIG. 4, the VRE comprises a virtual hospital corridor (VHC) 158 with a virtual object 160 comprising a virtual wheel chair 162, and a virtual floor 164 providing a virtual corridor, pathway, hall or hallway, virtual walls 166, virtual doors 168, and a virtual ceiling 170.

The person (subject or patient) 172 (FIG. 4) having the traumatic brain injury (TBI) or other neurocognitive disorder can be fitted with a safety harness 174 to face the virtual hospital corridor (VHC).

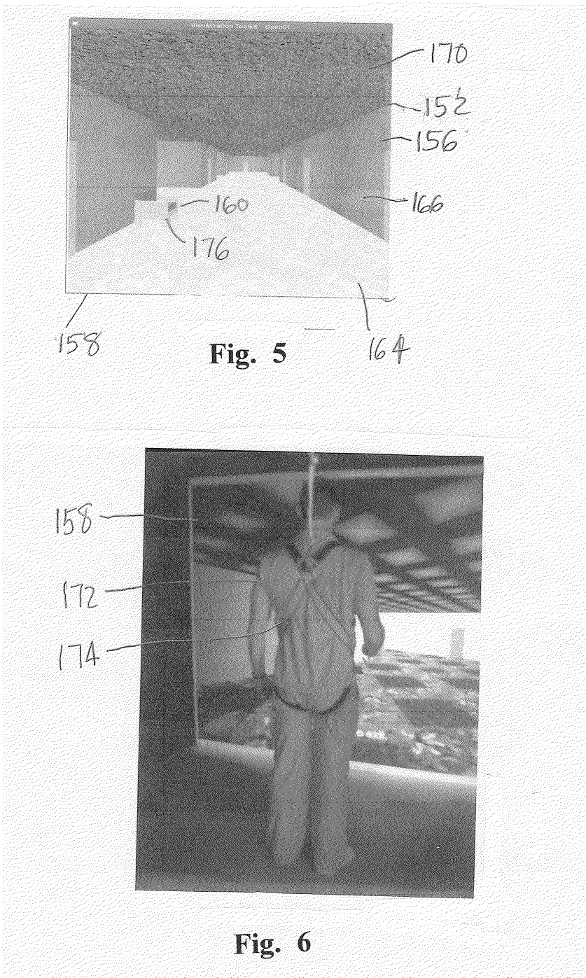

FIG. 5 illustrates the virtual hospital corridor with a virtual object 160 comprising a virtual chair 176.

FIG. 6 also illustrates the TBI person in the safety harness navigating the virtual hospital corridor.

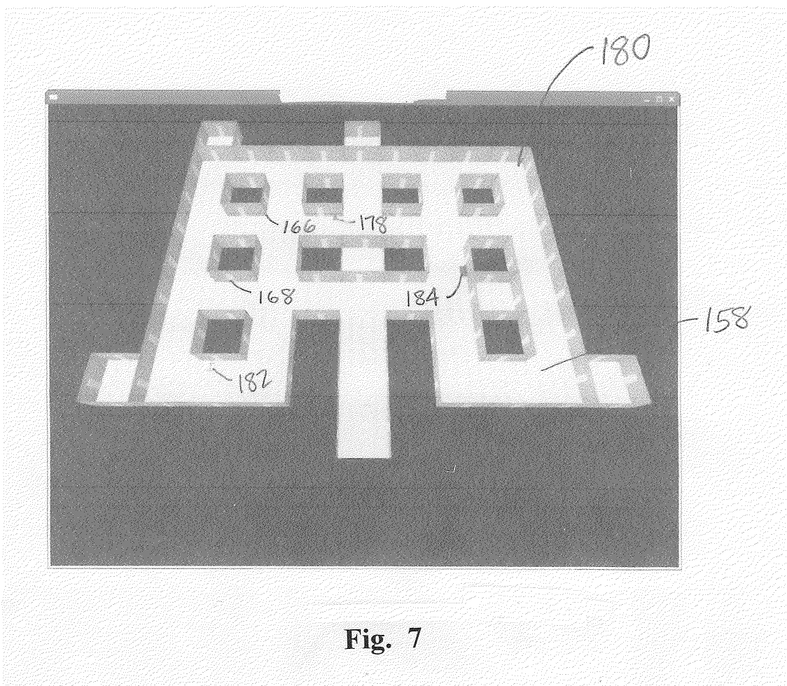

FIG. 7 is an overhead view of a virtual reality corridor system 180 illustrating multiple blocks with various combinations of walls as well as multiple props (virtual objects), such as a virtual hospital bed 182, a virtual couch 184 and a virtual chair 178, positioned in specific blocks.

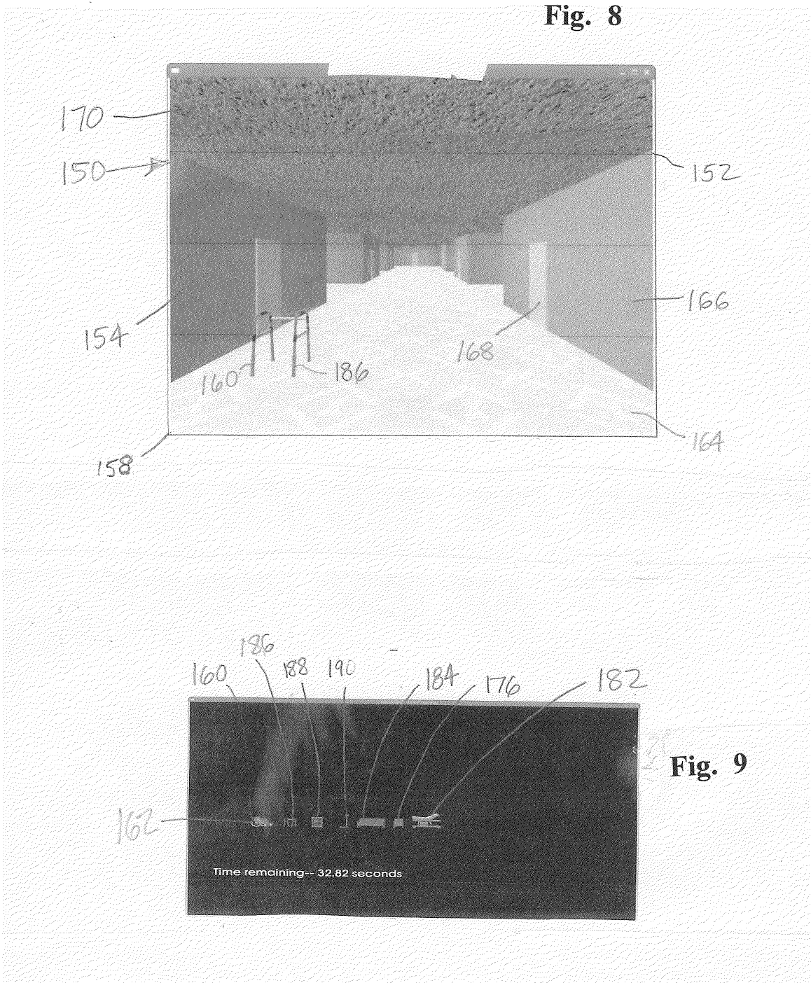

FIG. 8 illustrates the virtual hospital corridor with a virtual object 160 comprising a virtual walker 186.

FIG. 9 illustrates a virtual environment in which the TBI person (patient or subject) is asked to memorize virtual objects 160, such as seven virtual objects, e.g. a wheelchair 162, walker 186, sign 188, couch 184, hospital bed 182, and intravenous (IV) bag and tubing 190.

FIG. 10 illustrates a virtual environment with 14 virtual objects in which the TBI person is asked to select the seven virtual objects that appeared in the previous virtual hospital corridor of FIG. 8. The virtual objects 160 of FIG. 10 include the same virtual objects as seen in FIG. 9 as well as additional virtual objects, e.g. a virtual table, 192, screen 194, stand 196, a different color couch 198 and different color chair 200.

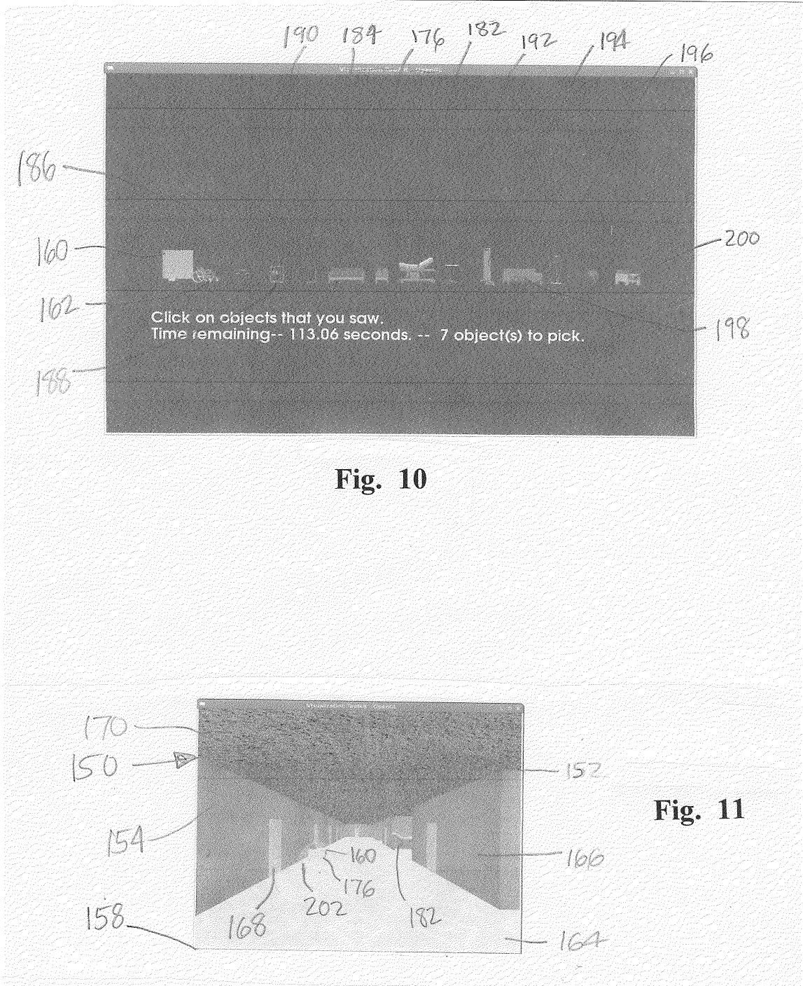

FIG. 11 illustrates a virtual corridor in which the TBI patient can select the previously seen virtual objects 160 under a transparent dome 202.

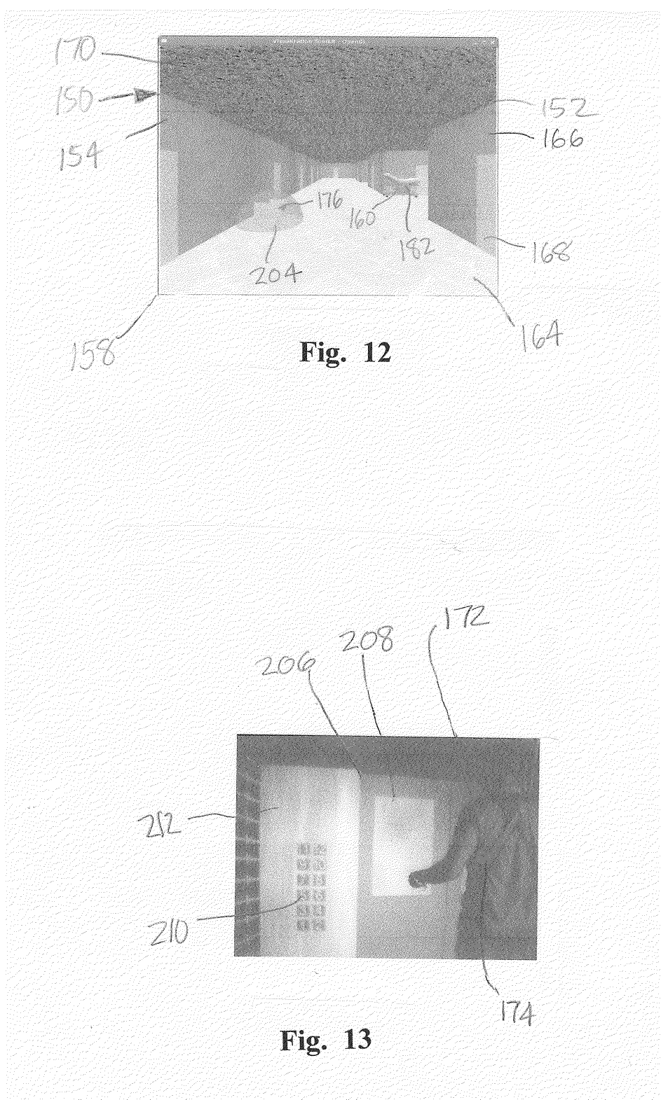

FIG. 12 illustrates the virtual corridor in which the TBI patient views the previously selected virtual objects 160 under a red dome 204.

FIG. 13 illustrates the TBI person in the safety harness 174 viewing the virtual elevator 206 with a virtual elevator door 208 and virtual buttons 210 on a virtual environment interactive panel 212 for the TBI person to select.

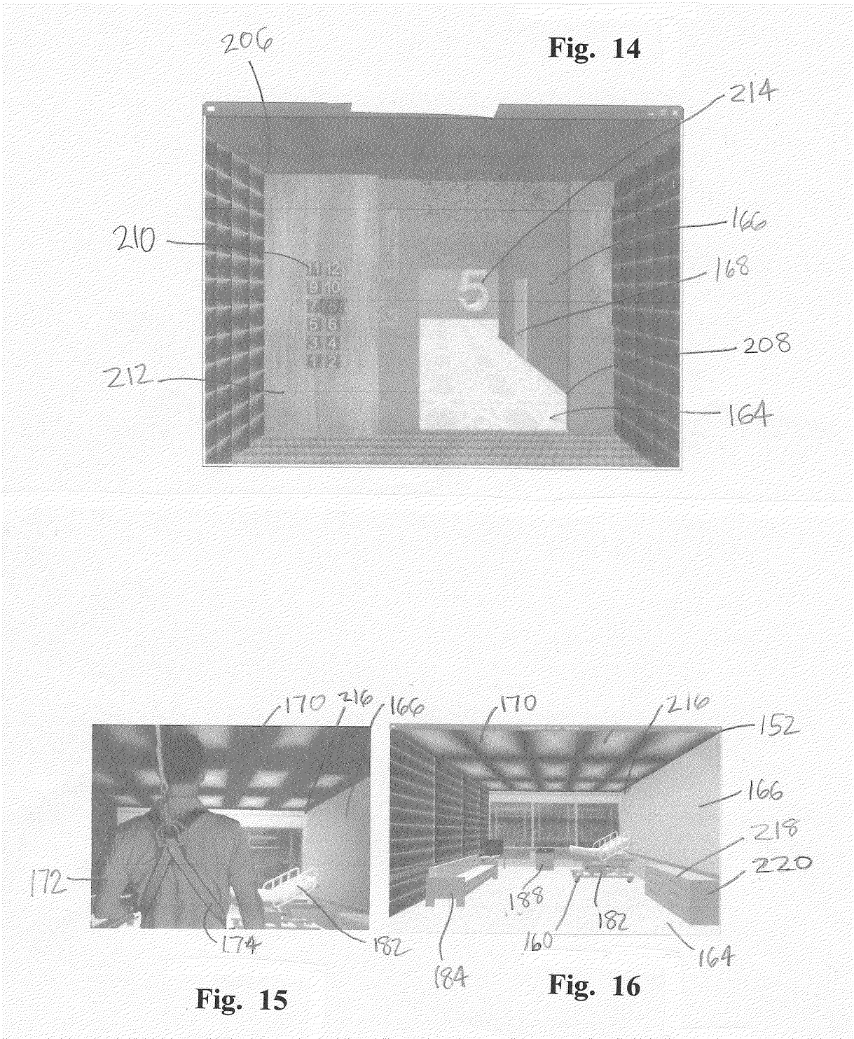

FIG. 14 illustrates a virtual elevator with the door open on the fifth floor 214 (Floor 5).

FIG. 15 illustrates the TBI person in the safety harness 174 viewing the virtual hospital room 216.

FIG. 16 illustrates the virtual hospital room with virtual objects 160 including a couch 184, chest 188, hospital bed 182, and other furniture 218 including a dresser 220.

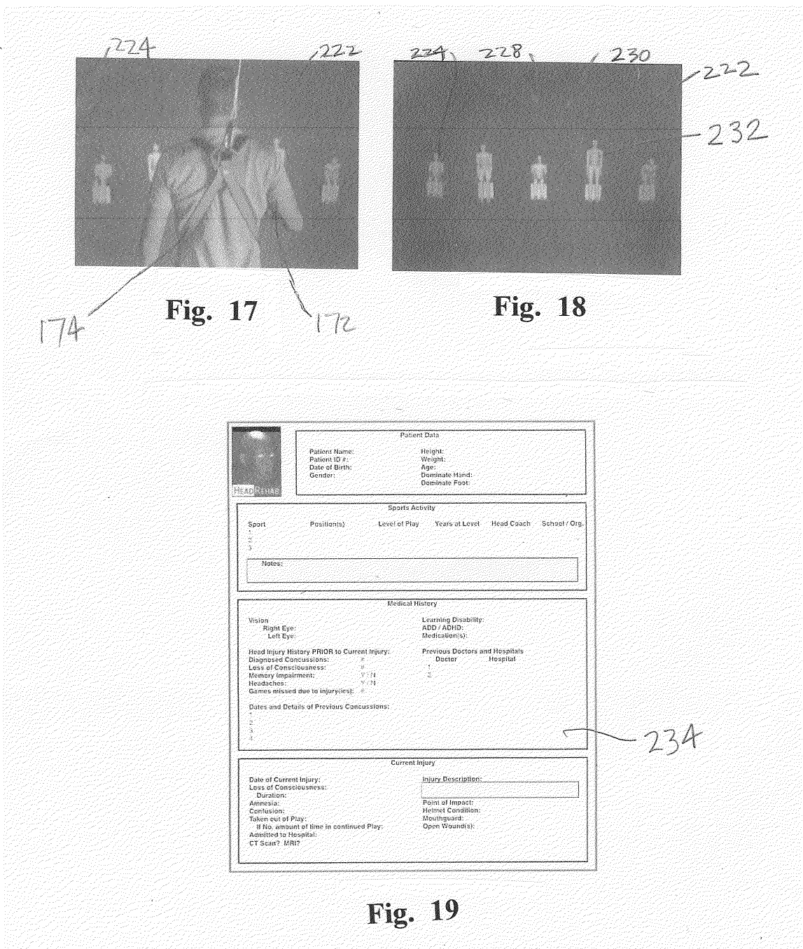

FIG. 17 illustrates the TBI person 172 in the safety harness 174 viewing the body module 222 with various positions 224 of a virtual persons' body 222

FIG. 18 illustrates the Body Module with position 224 of a virtual persons' body 222 moving from a sitting position 228 in a virtual chair 230 to a standing position 232 and vice versa.

FIG. 19 illustrates a report 234 generated by the output of the Report Module in cooperation with the CPU and printer or monitor.

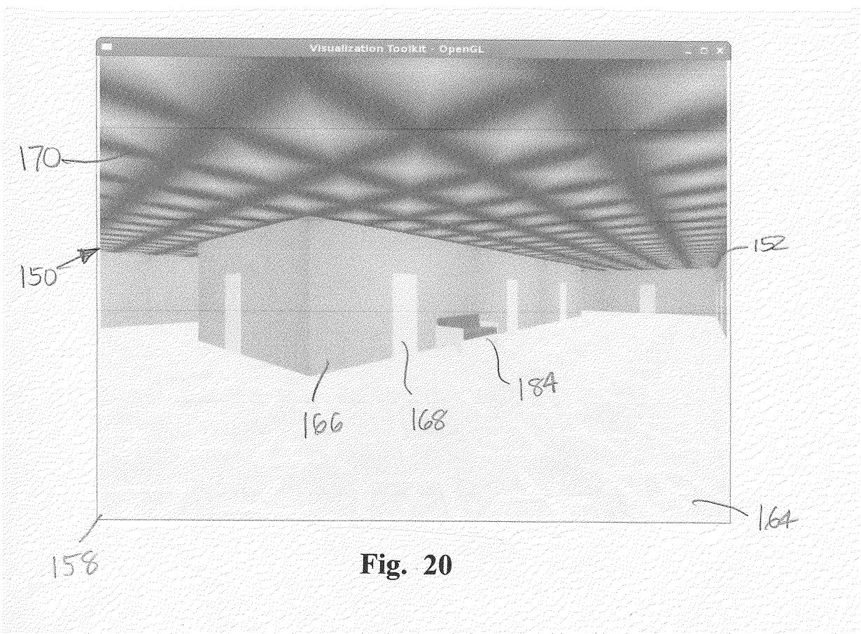

FIG. 20 is a front view of a virtual environmental screen snapshot comprising a virtual corridor showing virtual objects 160 (props) comprising a couch 184 and six virtual walls comprising a virtual ceiling, floor, front wall, back wall and left and right side walls.

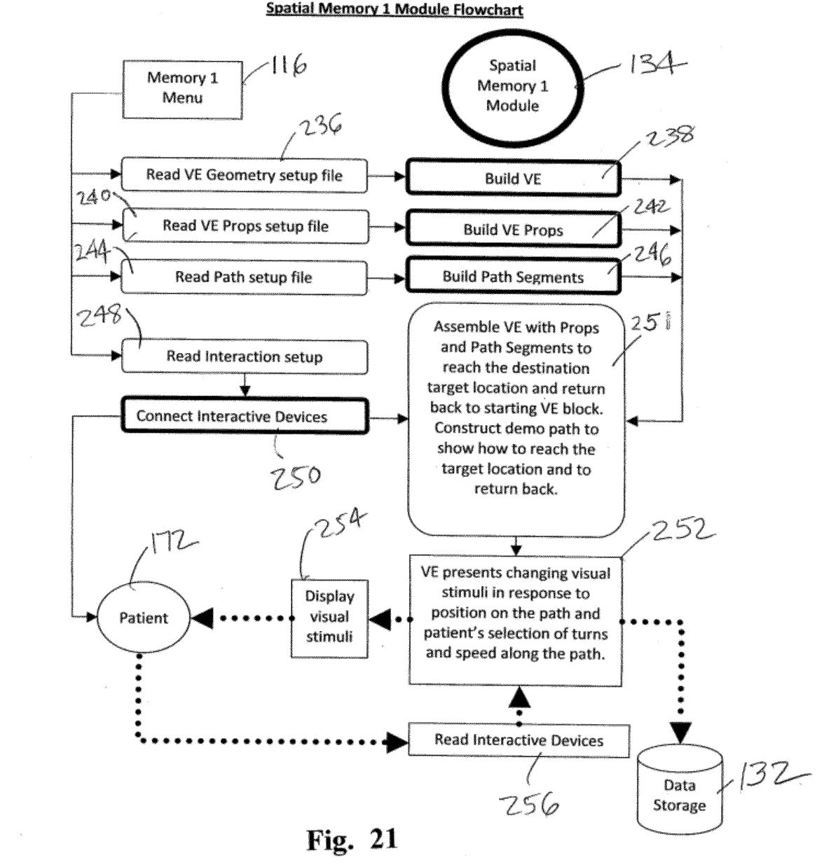

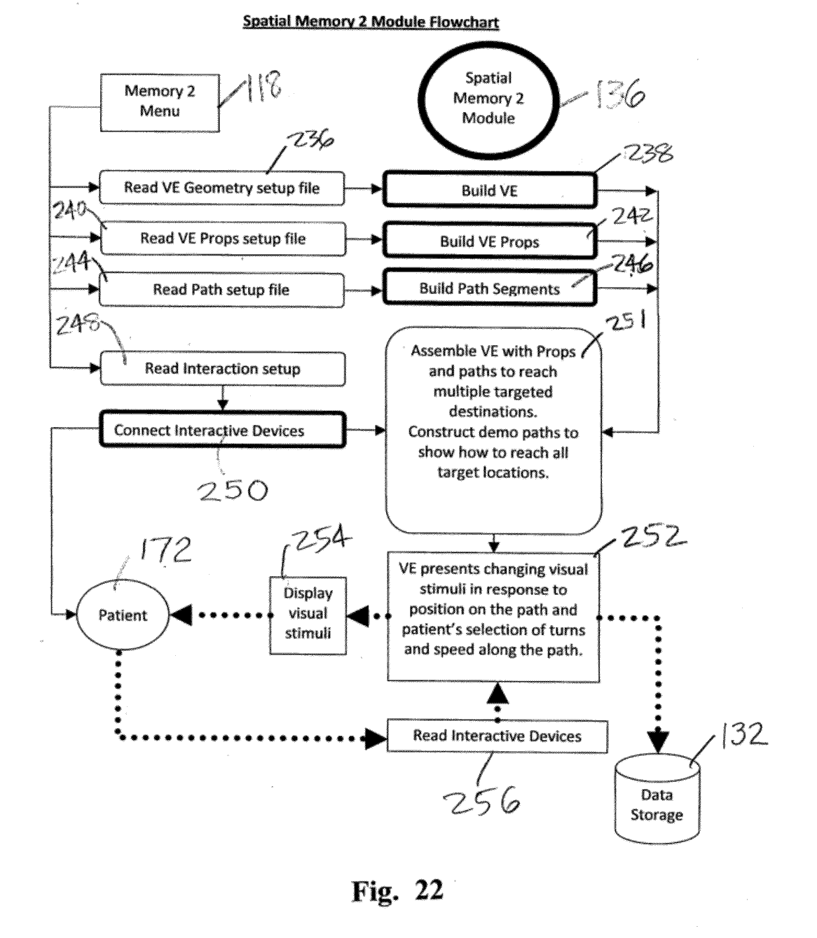

In the Spatial Memory 1 Module flowchart of FIG. 21, the Memory 1 Menu 116 is provided for use with and inputs into the Spatial Memory 1 Module 134. The Memory 1 Menu 116 includes: a Read Virtual Environment (VE) Geometry setup file 236 for use with and which inputs into the Build VE submodule 238; a Read VE Props setup file 240 for use with and which inputs into the Build VE Props submodule 242; a Read Path setup file 244 for use with and which inputs into the Build Path Segments submodule 246; and a Read Interaction setup 248 prior to and which inputs into the Connect Interactive Devices submodule 250. The Connect Interactive Devices submodule and submodules 238, 242 and 246 cooperate with and input the VE submodule 250 with data required to assemble the virtual objects (props) and path segments (corridor) and then construct a demonstration path to show the patient (person) how to reach the destination target location and return back to the starting VE block. The VE block 252 presents changing visual stimuli in response to the patient's (person's) current position on the path (corridor) and the patient's (person's) selection of turns and speed along the path and then displays the visual stimuli 254. As the displayed Virtual Environment changes, the patient 172 can make additional selections which are input into the Read Interactive Devices submodule 256 and subsequently to the VE block 252. This loop is repeated as the patient (person) 172 continues to interact with the software via the Interactive Devices 256. Output from the VE block is transmitted to Data Storage 132.

In the Spatial Memory 2 Module flowchart of FIG. 22, the Memory 2 Menu 118 is provided for use with and inputs into the Spatial Memory 2 Module 136. The Spatial Memory 2 Module flowchart of FIG. 22 is similar to the Spatial Memory 1 Module flowchart of FIG. 21, except for VE block 250 where the VE is assembled with props and paths to reach multiple targeted destinations.

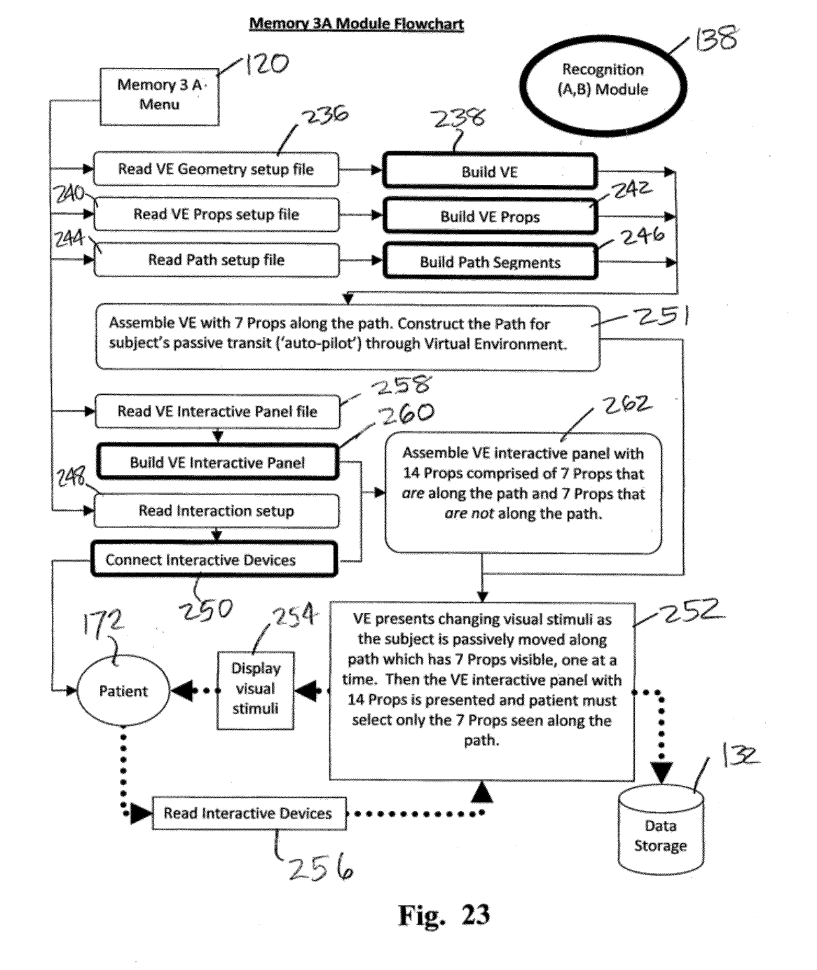

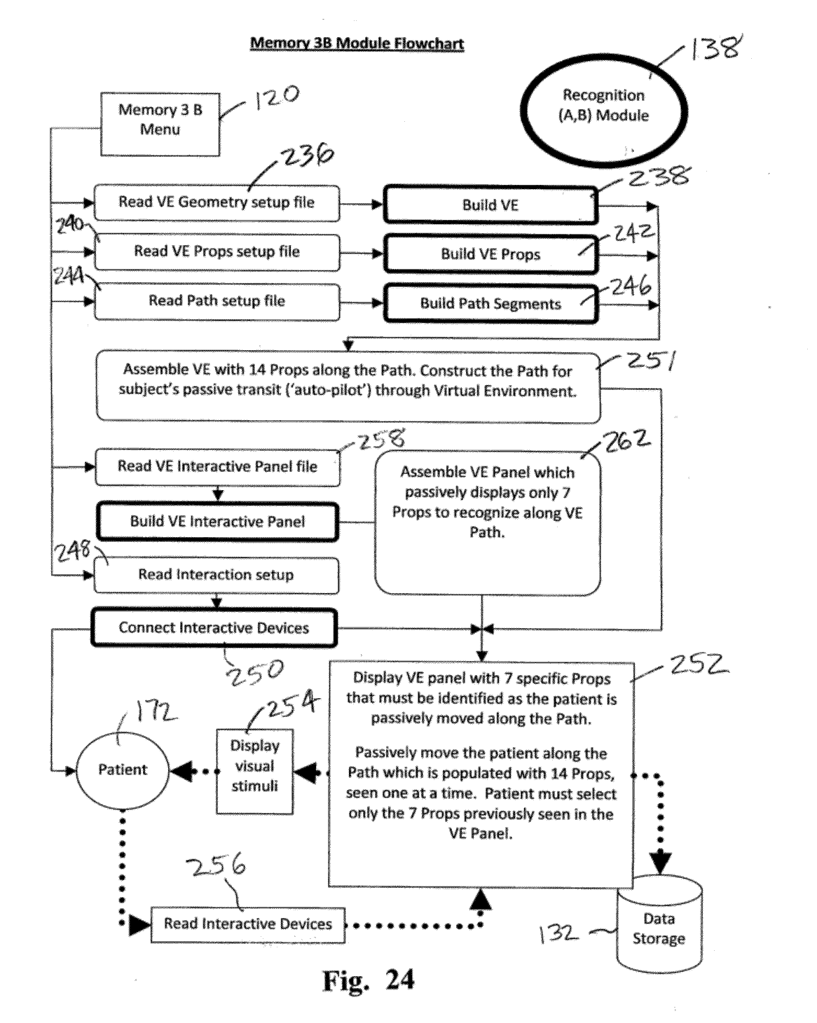

In the Memory 3A Module flowchart of FIG. 23, the Memory 3A Menu 120 is provided for use with and inputs into the Recognition (A, B) Module 138. The Memory 3A Module flowchart of FIG. 23 is similar to the Spatial Memory 1 Module flowchart of FIG. 21, but also has a Read VE Interactive Panel file 258 that inputs to a Build VE Interactive Panel 260. The Build VE Interactive Panel along with the Connect Interactive Devices submodule 250 inputs into the Assemble VE interactive panel submodule 262 which assembles seven virtual objects (props) that are along the virtual path (corridor) and seven virtual objects that are not along the path for a total of 14 virtual objects. The assembled VE Interactive Panel inputs into the VE block 252 which changes visual stimuli as the subject (person) is passively moved along the path to see seven visible virtual objects, one at a time. Afterwards, the VE Interactive Panel displays 14 virtual objects that are shown to the patient 172 who then selects the seven virtual objects that the patient saw along the path.

In the Memory 3B Module flowchart of FIG. 24, the Memory 3B Menu 120 is provided for use with and inputs into the Recognition (A, B) Module 138. The Memory 3B Module flowchart of FIG. 24 is similar to the Memory 3A Module flowchart of FIG. 23, except for the Assemble VE submodule 251 which assembles the virtual environment (VE) with 14 virtual objects (props) along the virtual path (corridor) and constructs the virtual path for the person's passive transit by auto-pilot through the virtual environment (VE). The Assemble VE Panel 262, in cooperation with the Display VE Panel 252, assembles and passively displays only seven virtual objects (props) for the TBI person (patient or subject) to recognize along the VE path. The Display VE Panel 252 moves the TBI person along the path to see the 14 virtual objects one at a time displayed and populated along the virtual path. The TBI person should select only the seven virtual objects that were previously seen by the TBI person on the VE panel.

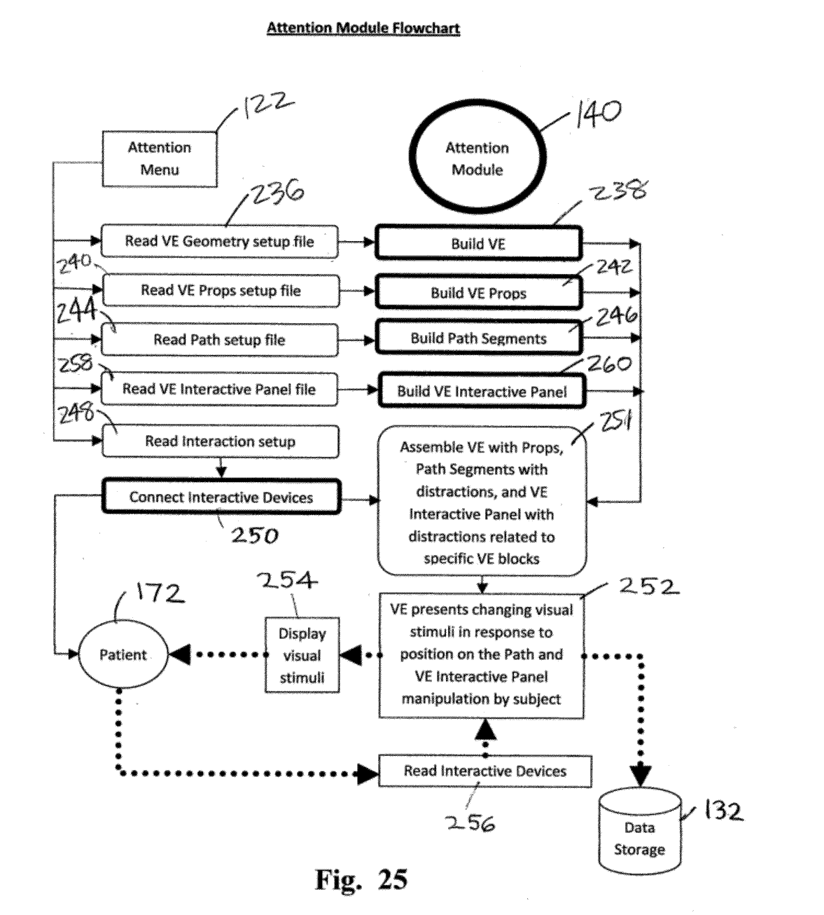

In the Attention Module flowchart of FIG. 25, the Attention Menu 122 is provided for use with and inputs into the Attention Module 140. The Attention Module flowchart of FIG. 25 is similar to the Memory 3A Module flowchart of FIG. 23, except the Assemble VE submodule 251 assembles the virtual environment (VE) with virtual objects (props), path segments, visual and/or audio distractions, as well as a VE Interactive Panel with audio and/or visual distractions, related to specific VE blocks. The VE submodule 252 changes visual stimuli in response to the position on the path (corridor) and VE Interactive Panel manipulation by the TBI person (subject or patient) 172.

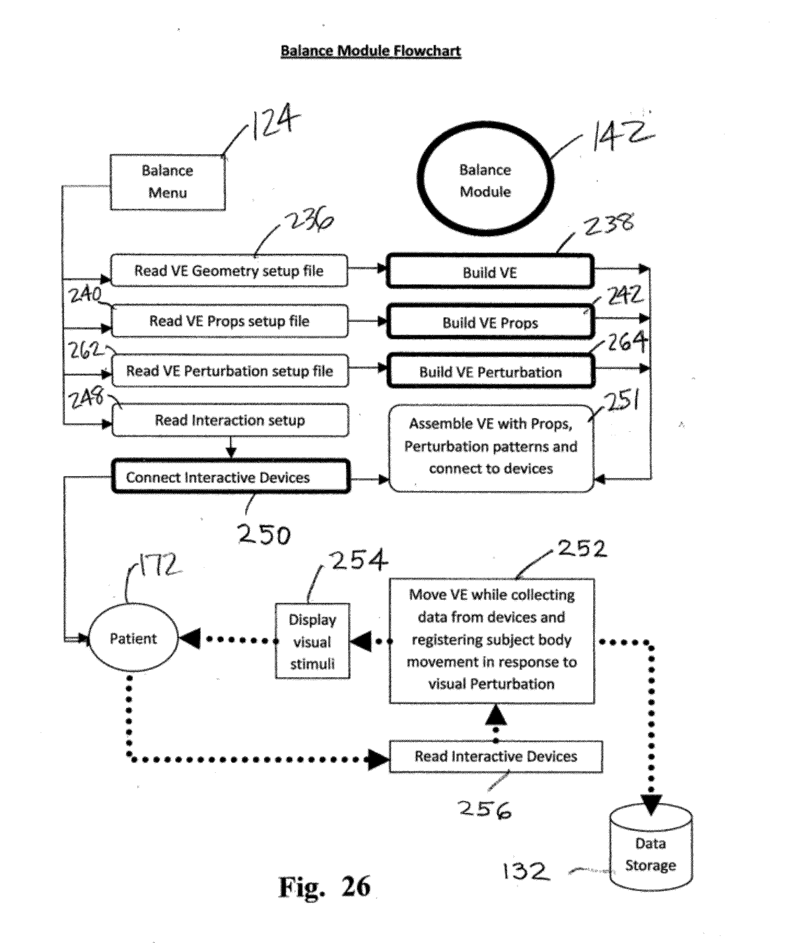

In the Balance Module flowchart of FIG. 26, the Balance Menu 124 is provided for use with and inputs into the Balance Module 142. The Balance Module flowchart of FIG. 26 is similar to the Spatial Memory 1 Module flowchart of FIG. 21, but has a Read VE Perturbation setup file 262 that inputs to a Build VE Perturbation submodule 264 instead of having a Read Path setup file 244 (FIG. 21) or Build Path Segments submodule 246. The Assemble VE module 251 assembles a virtual environment (VE) with virtual objects (props) and perturbation patterns and connects to the interactive devices. The VE display module 252 collects data from the interactive device and receives input and registers the movement of the TBI person in response to the visual perturbation.

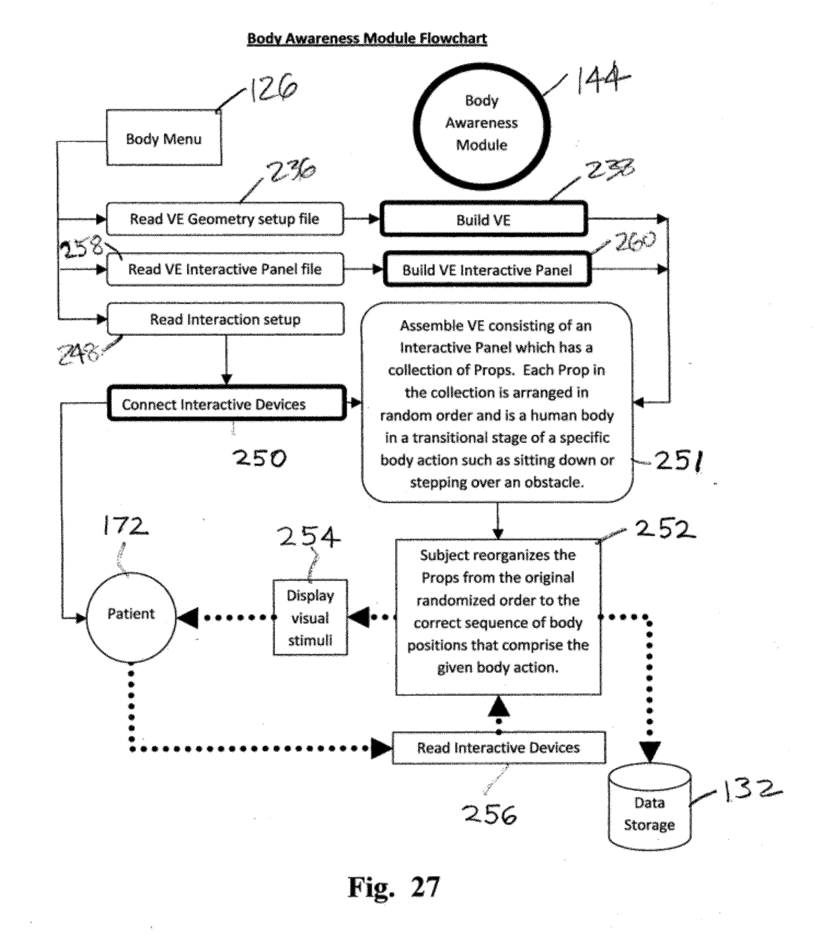

In the Body Awareness Module flowchart of FIG. 27, the Body Menu 126 is provided for use with and inputs into the Body Awareness Module 144. The Body Awareness Module flowchart of FIG. 27 is similar to the Attention Module flowchart of FIG. 25, except that it does not have a Read VE Props setup file 240 (FIG. 25), Read Path setup file 244, Build VE Props submodule 242 or Build Path Segments submodule 246. The Assemble VE submodule 251 assembles an interactive panel which has a collection of virtual objects (props). Each virtual object in the collection is arranged in random order and is a portion of a human body in a transitional stage of a specific body action such as sitting down, standing up or stepping over an obstacle. The VE module 252 receives input from the interactive devices when the TBI person (patient or subject) 172 reorganizes the virtual body positions (movements) from the original randomized order to the sequence of body positions the person believes to be correct.

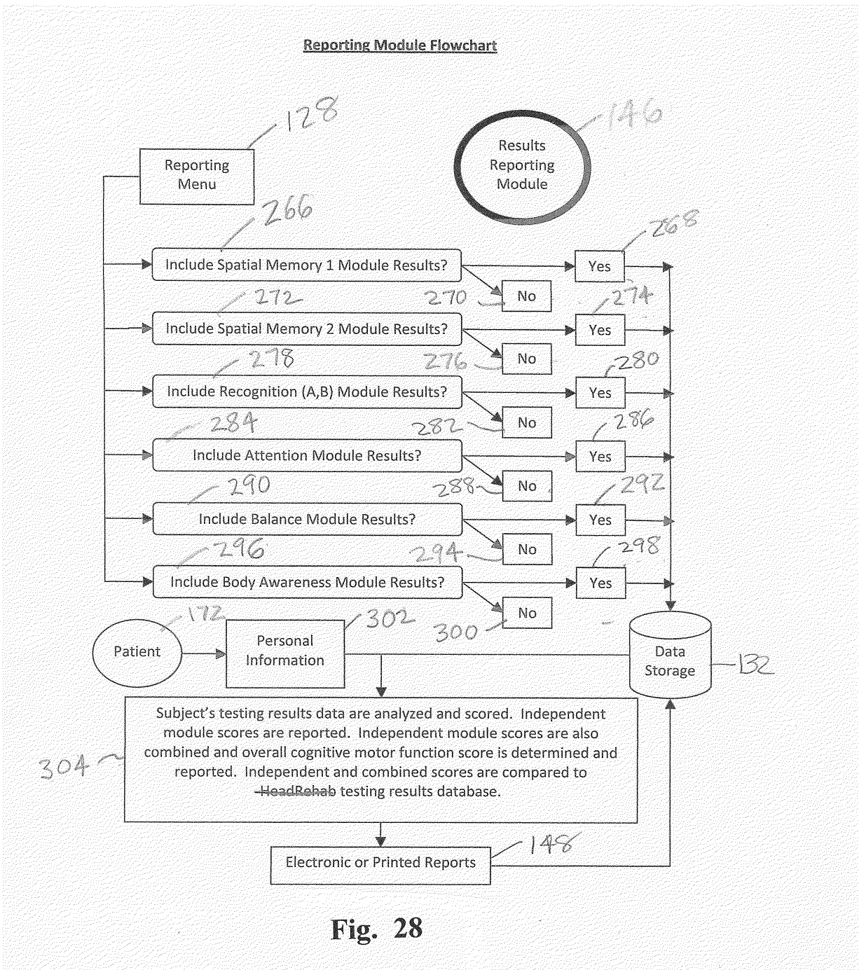

In the Reporting Module flowchart of FIG. 28, the Reporting Menu 128 is provided for use with and inputs into the Reporting Module 146. The Reporting Menu includes the following files (submenus) which allow the user to select the data transmitted to Data Storage 132. The Spatial Memory 1 Module results file 266 allows the Spatial Memory 1 Module results data to be transmitted via the Yes (positive or affirmative) Spatial Memory 1 submodule 268 to Data Storage 132 or prevents the Spatial Memory 1 Module results data from being transmitted via the No (negative) Spatial Memory 1 submodule 270 to Data Storage 132. The Spatial Memory 2 Module results file 272 allows the Spatial Memory 2 Module results data to be transmitted via the Yes (positive or affirmative) Spatial Memory 2 submodule 274 to Data Storage 132 or prevents the Spatial Memory 2 Module results data from being transmitted via the No (negative) Spatial Memory 2 submodule 276 to Data Storage 132. The Recognition (A, B) Module results file 278 allows the Recognition (A, B) Module results data to be transmitted via the Yes (positive or affirmative) Recognition (A, B) submodule 280 to Data Storage 132 or prevents the Recognition (A, B) Module results data from being transmitted via the No (negative) Recognition submodule 282 to Data Storage 132. The Attention Module results file 284 allows the Attention Module results data to be transmitted via the Yes (positive or affirmative) Attention submodule 286 to Data Storage 132 or prevents the Attention Module results data from being transmitted via the No (negative) Attention submodule 288 to Data Storage 132. The Balance Module results file 290 allows the Balance Module results data to be transmitted via the Yes (positive or affirmative) Balance submodule 292 to Data Storage 132 or prevents the Balance Module results data from being transmitted via the No (negative) balance submodule 294 to Data Storage. The Body Awareness module results file 296 allows the Body Awareness Module results data to be transmitted via the Yes (positive or affirmative) Body Awareness submodule 298 to Data Storage 132 or prevents the Body Awareness Module results data from being transmitted via the No (negative) Body Awareness submodule 300 to Data Storage 132. The TBI person (patient or subject) 172 can give Personal Information 302 which is inputted to Scoring submodule 304 and later is stored in Data Storage 132. The Scoring submodule 304 receives input from Data Storage 132 and electronically analyzes and scores the results data from the independent modules. Independent module scores can also be combined in the scoring module. The overall cognitive motor function score can be electronically determined in the scoring module. The independent and combined scores are electronically compared to a testing results database. The output from the scoring modules can be transmitted and sent to generate Reports 148 such as electronic reports or printed reports via the printer.

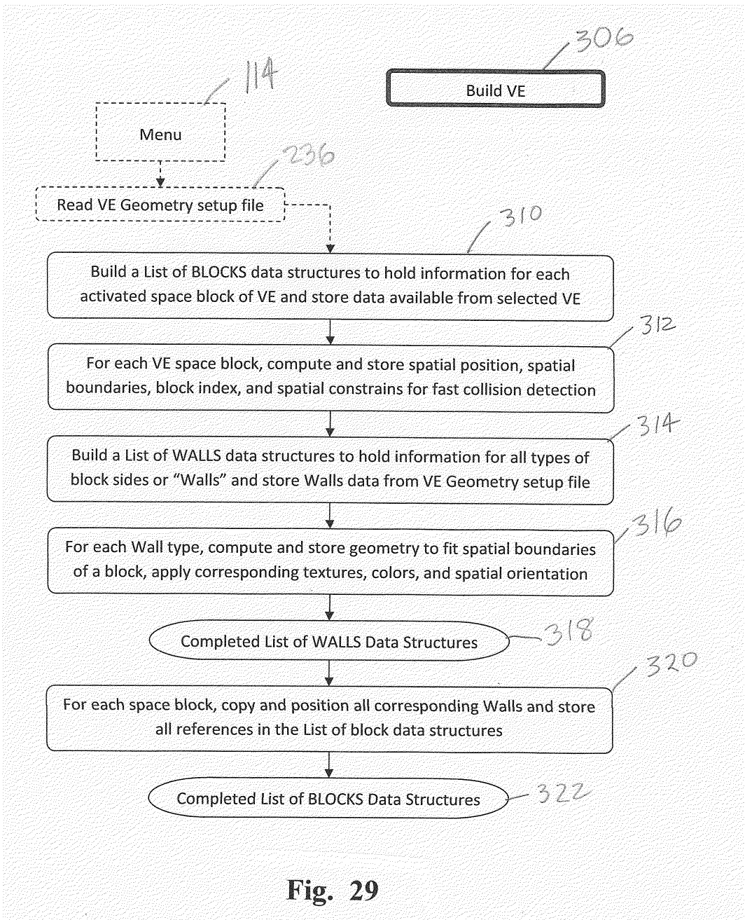

In the Build Virtual Environment (VE) flowchart of FIG. 29, the Build VE Module 306 has a Menu 114 and Read VE Geometry setup file 236 which inputs into a Build a List of Blocks submodule 310 that electronically builds a list of blocks data structures to hold information for each activated space block of VE as well as store data available from a selected VE. The Compute and Store Spatial Positions submodule 312 receives input from the Build a List of Blocks submodule and computes and electronically stores the spatial position spatial boundaries, block index and spatial constraints for fast collision detection for each VE space block. The Build a List of Walls submodule 314 receives input from the Compute and Store Spatial Positions submodule 312 and electronically builds a list of walls data structures to hold information for all types of block sides or walls, as well as stores walls data from the VE Geometry setup file 236. The Geometry submodule 316 receives input from the Build a List of Walls and computes and electronically stores geometry to fit spatial boundaries of a block for each wall type as well as apply corresponding textures, colors and spatial orientation. The List of Walls Data Structure file 318 receives input from the Geometry submodule 316 and electronically completes the list of walls data structures. The Storage submodule 320 receives input from the List of Walls Data Structure file 318 and electronically copies and positions all corresponding walls for each space block and electronically stores all references in the competed electronic List of Blocks Data Structures 322.

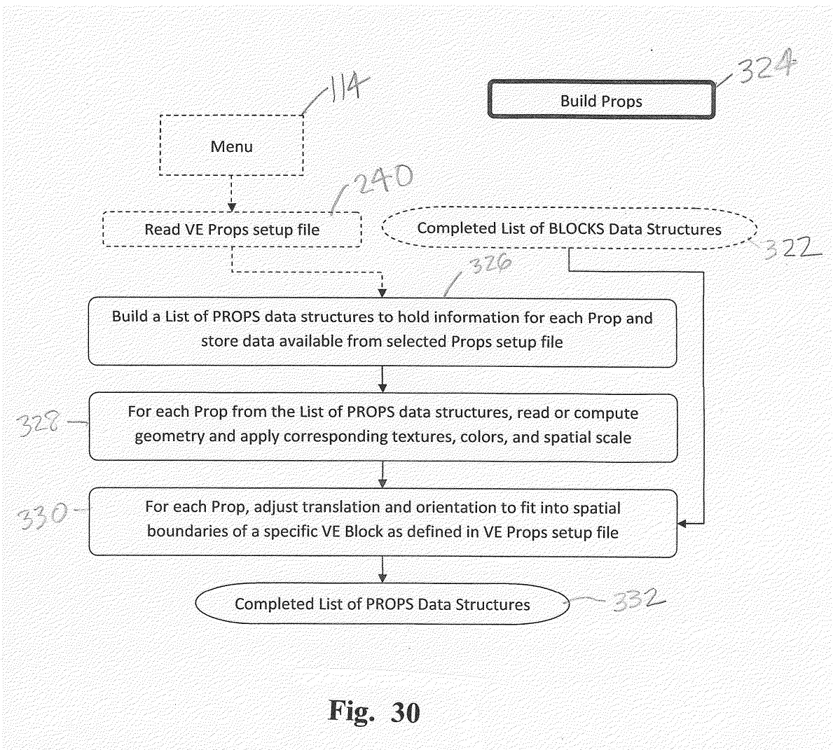

In the Build Virtual Props flowchart of FIG. 30, the Build Props Module 324 has a Menu 114 and a Read VE Props setup file 240 which inputs into a Build a List of Props submodule 326 that electronically builds a list of props (virtual objects) data structures to hold information for each prop (virtual object) as well as stores data available from a selected props setup file. The Props Geometry submodule 328 receives input from the Build a List of Props submodule 326 and electronically reads and/or computes the geometry for each prop from the list of props data structures, as well as electronically applies corresponding textures, colors and spatial scale. The Adjustment submodule 330 receives input from the Props Geometry submodule 328 and the completed List of Blocks Data Structures 322. The Adjustment submodule 330 electronically adjusts the translation and orientation for each prop to fit into spatial boundaries of a specific VE block as defined in the VE Props setup file 240 to provide a Completed List of Props Data Structures 332.

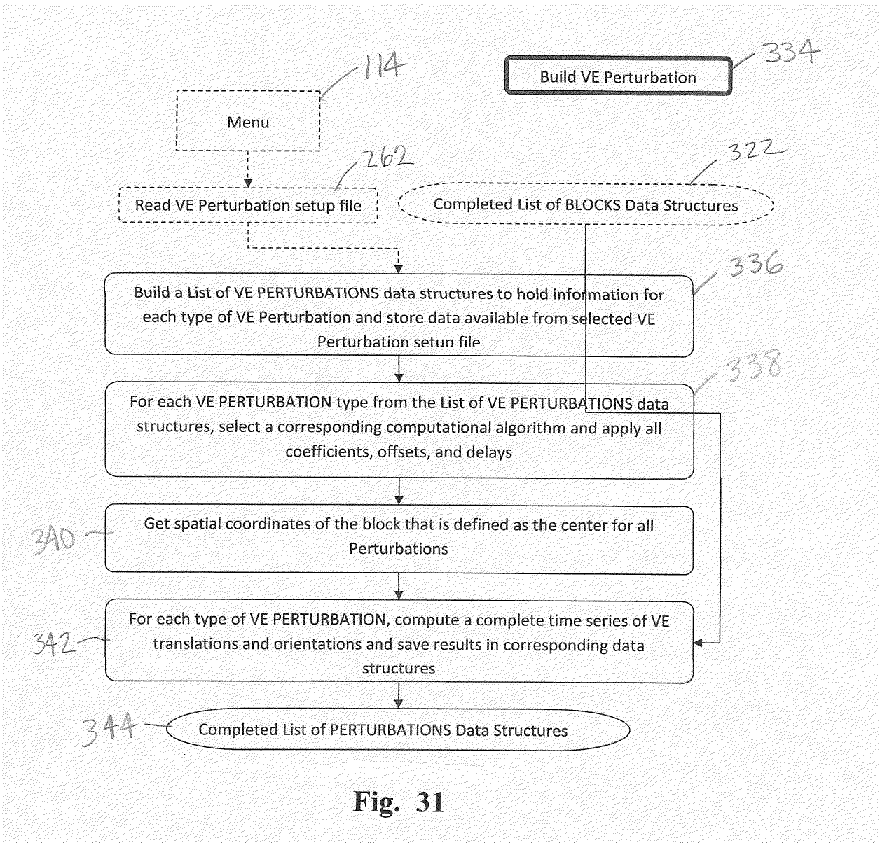

In the Build Virtual Environment (VE) Perturbation flowchart of FIG. 31, the Build VE Perturbation Module 334 has a Menu 114 and Read VE Perturbation setup file 262 which inputs into a Build a List of VE Perturbations submodule 336 that electronically builds a list of VE perturbations data structures to hold information for each type of VE perturbation as well as electronically stores data available from selected VE Perturbation setup file 262. The Selection submodule 338 receives input from the Build a List of VE Perturbations submodule 336 and electronically selects a corresponding computational algorithm for each VE perturbation type from the list of VE perturbations data structures and thereafter electronically applies all coefficients, offset and delays. The Spatial Coordination submodule 340 receives input from the Selection submodule 338 and electronically retrieves spatial coordinates of the block that is defined as the center for all perturbations. The Perturbation Computation submodule 342 receives input from the Spatial Coordination submodule 340 and the Completed List of Blocks Data Structures 322. The Perturbation Computation submodule 342 electronically computes a complete time series of VE translation and orientations as well as saves results in corresponding data structures for each type of VE perturbation to provide a Completed List of Perturbations Data Structures 344.

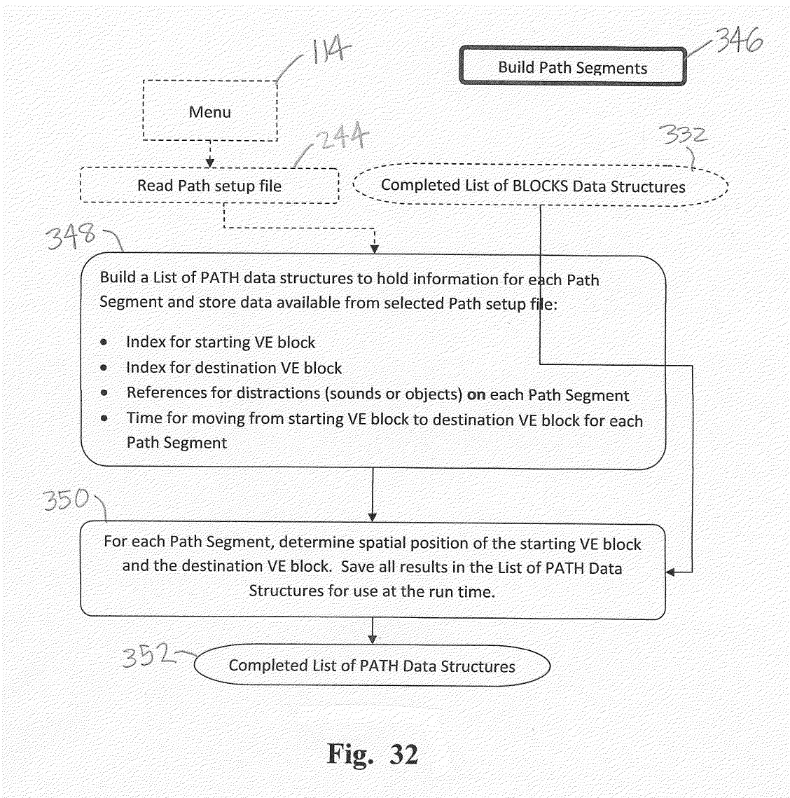

In the Build Path Segments flowchart of FIG. 32, the Build Path Segments Module 346 has a Menu 114 and Read Path setup file 244 which inputs into a Build a List of Path Segments submodule 348 that electronically builds a list of path data structures to hold information for each path segment as well as electronically store data available from a selected path setup file. The Build a List of Path Segments submodule 348 also provides for an electronic index for the starting VE block, an electronic index for the destination VE block, electronic references for audio and visual distractions, (e.g. sounds or virtual objects) on each path segment, and time for moving from the starting VE block to the destination VE block for each path segment. The Path Segment Spatial Position submodule 350 receives input from the Build a List of Path Segments submodule 348 as well as the Completed List of Blocks Data Structures 332. The Path Segment Spatial Position submodule 350 electronically determines the spatial position of the starting VE block and the destination of the VE block for each path segment (corridor). The Path Segment Spatial Position submodule 350 also electronically saves all results in the Completed List of Path Data Structure 352 for use at the run time.

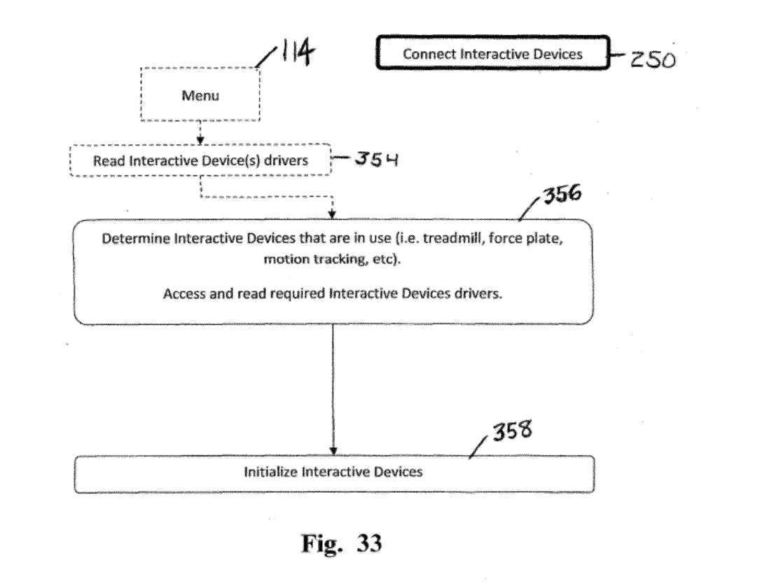

In the Connect Interactive Devices flowchart of FIG. 33, the Connect Interactive Devices subroutine 250 has a Menu 114 and Read Interactive Device(s) drivers 354 which inputs into a Determine Interactive Devices submodule 356 that are in use, (e.g. treadmill, force plate, motion tracking system) as well as electronically accesses and reads the required interactive device drivers. An Initialize submodule 358 receives input from the Determine Interactive Devices submodule 356 and electronically initializes the interactive devices.

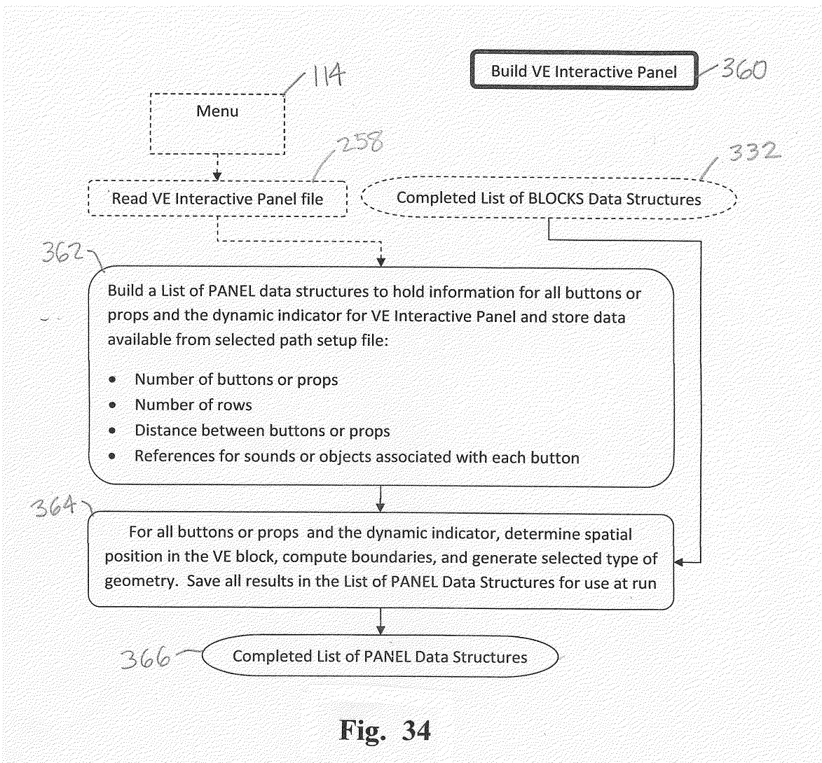

In the Build Virtual Environment (VE) Interactive Panel flowchart of FIG. 34, the Build VE Interactive Panel Module 360 has a Menu 114 and Read VE Interactive Panel file 258 which inputs into a Build a List of Panel Data Structures submodule 362 that electronically builds a list of panel data structures to hold information for all buttons or props (virtual objects) and provides a dynamic indicator for the VE interactive panel as well as electronically stores data available from the select path setup file. The Build a List of Panel Data Structures submodule 362 also provides for the number of buttons or props, the number or rows, the distance between buttons or props, and references for sounds or virtual objects associated with each button. The Interactive Spatial Geometry submodule 364 receives input from the Build a List of Panel Data Structures submodule 362 and the Completed List of Blocks Data Structures 332. The Interactive Spatial Geometry submodule 364 also electronically determines spatial positions in the VE block, electronically computes boundaries and electronically generates selected type of geometry for all buttons or props as well as the dynamic indicator to provide a Completed List of Panel Data Structures 366.

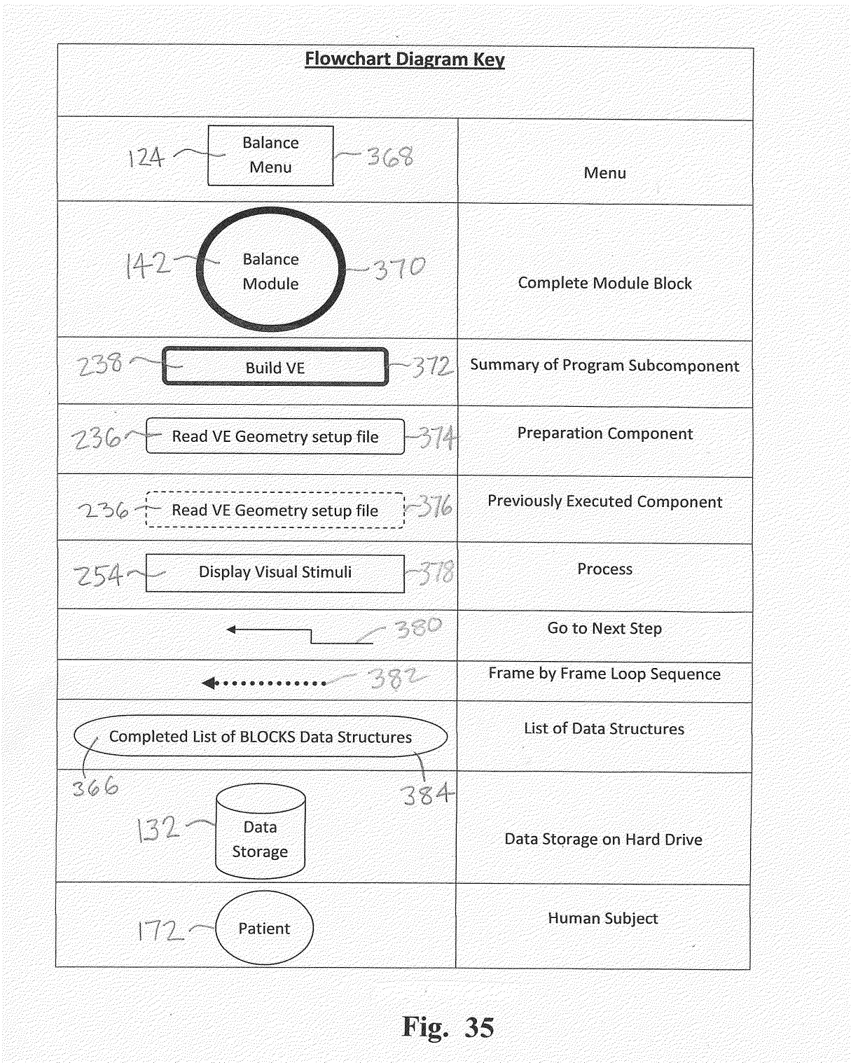

FIG. 35 is a Flowchart Diagram Key for the diagnostic (assessment) and treatment (rehabilitation) processes. The solid rectangle 368 with square (perpendicular) corners around (about) the Balance Menu 124 indicates it is part of the Menu. The ellipse 370 around the Balance Module 142 indicates it is a Complete Module Block. The bold solid elongated rectangle 372 with solid rounded (curved) corners around the Build VE submodule 238 indicates it is a Summary of the Program Subcomponent. The solid elongated rectangle 374 with solid rounded corners around the Read VE Geometry setup file 236 indicates it is a Preparation Component. The dotted elongated rectangle 376 with dotted rounded (curved) corners around the Read VE Geometry setup file 236 indicates it is a Previously Executed Component. The solid elongated rectangle 378 with solid square corners around the Display Visual Stimuli 254 indicates it is a Process. The solid line 380 with an arrow indicates to Go to the Next Step. The dotted line 382 with the arrow indicates it is a Frame by Frame Loop Sequence. The elongated oval 384 around the Completed List of BLOCKS Data Structures 366 indicates it is a List of Data Structures. Data Storage 132 can be stored on a hard drive or other electronic storage. The Patient 172 is a human being (subject, patient or person)

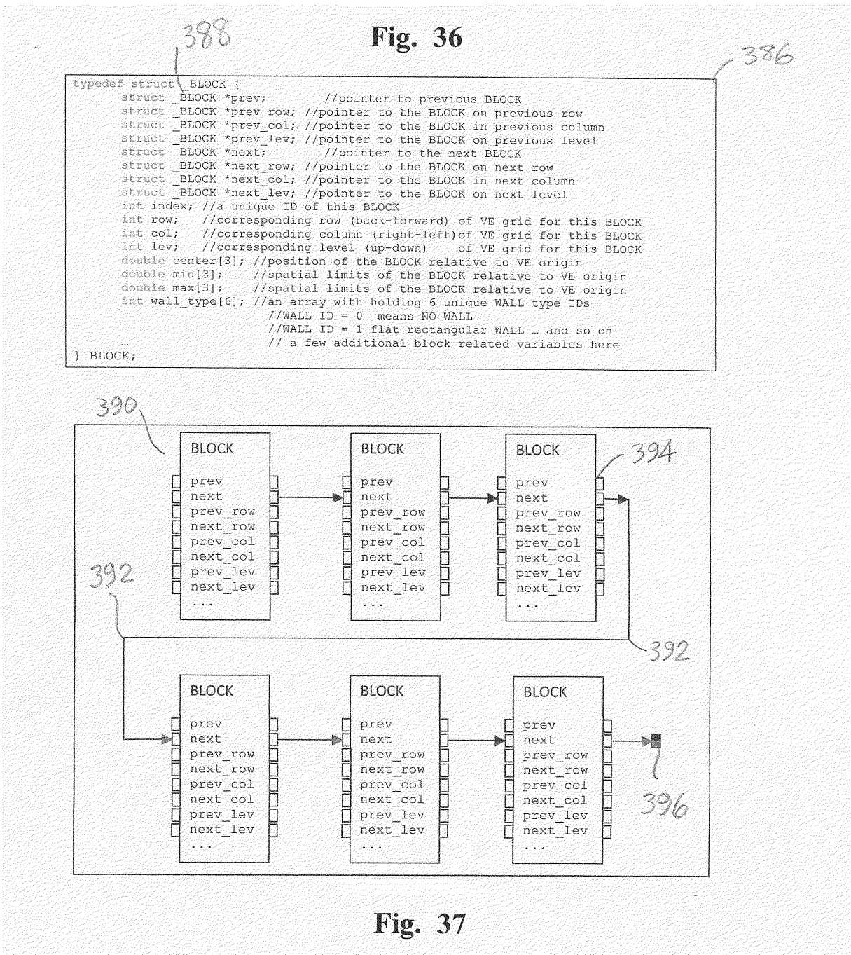

FIG. 36 is a chart of a truncated piece of code 386 defining a block data structure 388 for use in the TBI or other neurological disorder diagnostic (assessment) process.

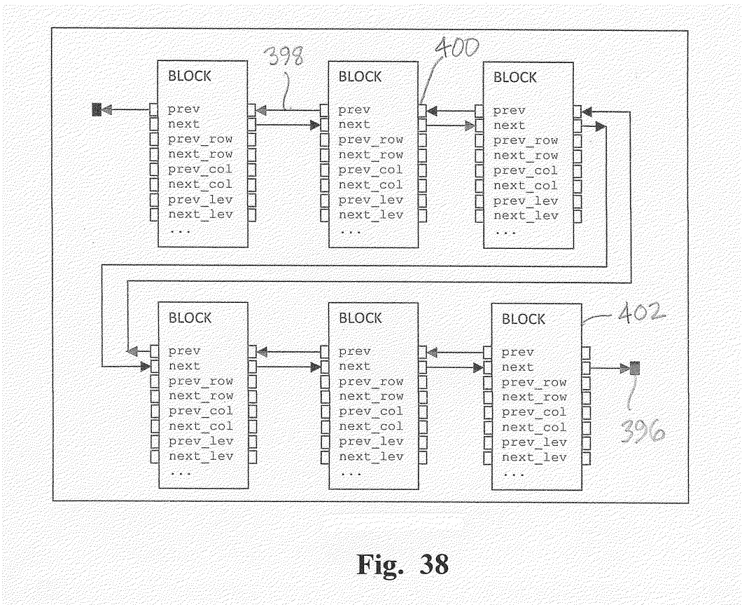

FIG. 37 is a diagram of a model representing a list 390 or linked list 392 of block data structures 394 where the solid block 396 represent a null pointer terminating the list for use in the TBI diagnostic (assessment) process.

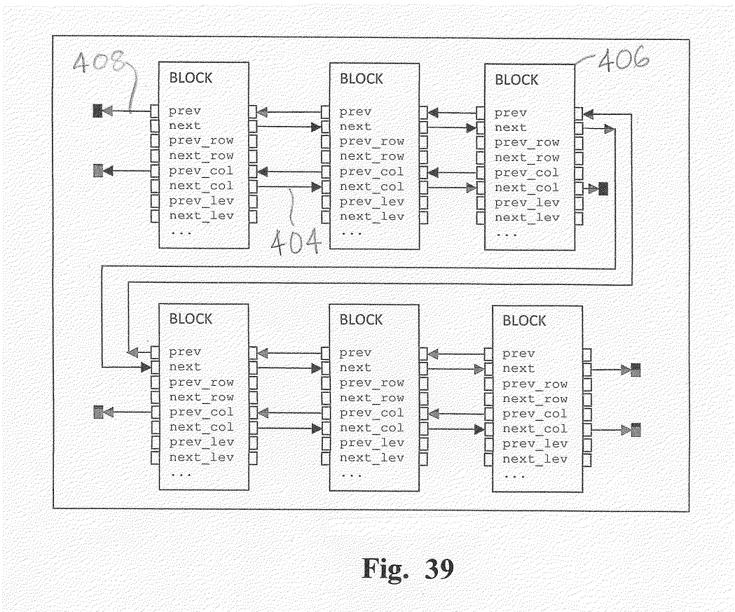

FIG. 38 is a diagram of a model representing a doubly linked list 398 of six block data structures 400 to hold the data for building a virtual environment with six blocks 402 positioned in two rows and three columns for use in the TBI diagnostic process.

FIG. 39 is a diagram of a model representing a complex linked list 404 of six block data structures 406 with additional links 408 providing fast access to an adjacent block in the next or previous column for use in the TBI diagnostic (assessment) process.

The novel process (method) can be used with a person (subject or patient) having a traumatic brain injury (TBI) and/or other neurological disorder(s) and provides a patient-friendly process for diagnosing (assessing) and treating (rehabilitating) impairment caused by TBI and/or other neurological disorder(s). The novel process can comprise: generating a three-dimensional (3D) virtual reality environment (VRE) comprising at least one scene with moveable virtual images and scenes with a central processing unit (CPU) in conjunction with at least one module; electronically displaying the VRE on a screen to a person with a traumatic brain injury (TBI) and/or other neurological disorder(s); and identifying a task to be performed by the person in the VRE. The person with the TBI and/or other neurological disorder(s) can electronically perform the task with an electronic interactive communications device. Thereafter, interactive communications comprising the person's responses and performance of the task can be electronically inputted to the CPU with the electronic interactive communications device.

The performance of the person with the TBI and/or other neurological disorder(s) can be electronically evaluated in the CPU based upon the electronically inputted interactive communications to the CPU. Furthermore, the performance data of the person with the TBI can be electronically compared in the CPU with the person's prior performance data or normal performance data from a data base. The comparison data can be electronically scored in the CPU. The person's impairment and extent of the TBI or other neurocognitive disorder can also be electronically assessed in the CPU and a deficiency of at least one of the person's functions can be electronically determined in the CPU. The function can comprise one or more cognitive functions, such as memory, recall, recognition, attention, and spatial awareness, and/or a motor function, such balance. Advantageously, the performance data, comparison data, and score from the CPU can be electronically reported, such as electronically displaying the score and comparison data from the CPU on the screen and/or printing the score and comparison data from the CPU in a printed report. The score and reported data can be used to help rehabilitate the person with the TBI.

The CPU can comprise, but is not limited to: a computer, laptop, desktop computer, portable computer, computer workstation, microprocessor, computer system, iPad, tablet computer, wireless computer, wired computer, netbook, electronic communications device, portable networking device, internet communication device, mobile phone, flip phone, camera phone, clamshell phone, radio telephone, cellular phone, smart phone, tablet phone, portable media payer (PMP), personal digital assistant (PDA), wireless e-mail device, handheld electronic device, mobile electronic device, video game device, video game console, video game player, electronic amusement device for use with a television or monitor, video gaming device, or a combination of any of the preceding.

The interactive communications device can comprise, but is not limited to: an electronic joystick, electronic mouse, three-dimensional (3D) electronic mouse, electronic controller, navigation controller, handheld controller, fMRI-compatible mouse, wireless controller, wired controller, voice activated controller, video game controller, inputting device, key pad, screen pad, touch pad, keyboard, treadmill, motion tracking device, force sensing device, force platform, force plate, wireless interactive communications, real-time motion tracking magnetic and ultra-sound systems, wands or a combination of any of the preceding.

The screen can comprise one or more of the following: a portable screen, touch screen, computer screen, touch pad, display, monitor, wall, shade, liquid crystal screen, projection screen, video projection screen, video screen, television, high definition television, 3D television, virtual reality goggles and virtual reality headset.

The module can comprise at least one of the following modules: a memory module, spatial memory module, recognition module, object recognition module, attention module, body module; body awareness module, and results reporting module.

The task can comprise one or more of the following tasks, but is not limited to: object recognition, virtual navigation, virtual walking, virtual steering, spatial navigation, object navigation, spatial memory, kinesthetic imagery, virtual arrangement of images, standing, balance and memorizing virtual objects.

The virtual images can comprise virtual 3D images including one or more of the following, but is not limited to: a virtual elevator, virtual elevator buttons, virtual corridor, virtual hospital corridor, virtual body, virtual hospital room, virtual bathroom, virtual door, virtual hall, virtual wall, virtual room, virtual pathway, virtual object, virtual sign, virtual picture, virtual bed, virtual floor, virtual cart, virtual stretcher, virtual person, virtual table, virtual positions of a person's body, virtual furniture, virtual walker, virtual wheelchair, virtual hospital bed, virtual couch and virtual stand.

For safety reasons it is preferred to securely place a safety harness on the person with TBI and/or other neurological disorder(s) before the person with the TBI and/or other neurological disorder(s) experiences the virtual reality environment and/or performs the task.

In the diagnostic (assessment) and rehabilitative process, the task can include, but is not limited to: virtual navigation, virtual walking, spatial navigation, virtual object selection, virtual object manipulation or combinations thereof for use in conjunction with a memory module, spatial memory module, recognition module or object recognition module. The virtual 3D images can include: a virtual hospital corridor, virtual hospital room, virtual room, virtual pathway, virtual floor, virtual bed, virtual bathroom and/or virtual object panel. The task can be electronically performed by virtually arriving at the virtual destination.

In the diagnostic (assessment) and rehabilitative process, the person with the TBI and/or other neurological disorder(s) can electronically perform the task by identifying virtual 3D objects in the virtual hospital corridor in cooperation with the object recognition module or by identifying the correct order of virtual body positions in cooperation with the body awareness module.

In the diagnostic and rehabilitative process, in conjunction with the balance module or attention module, the person with the TBI performing the task can stand on an interactive communications device which can comprise a moveable and tiltable force sensing device, such as a force platform or force plate, that is electronically hardwired to the CPU and/or connected by wireless communications with the CPU. The person can also communicate with the software via an interactive device. The person with the TBI and/or other neurological disorder(s) can view on the screen virtual 3D images such as a virtual elevator, virtual door, virtual panel with virtual elevator buttons, virtual floors and virtual hospital room. One such attention task can be performed by the person with the TBI by recognizing floor numbers in response to virtual movement of the virtual elevator. The VRE on the screen can comprise multiple virtual environments and can include one or more distractions for the person with TBI performing the task. The distractions can comprise electronic visual distractions on the screen and/or electronic audible distractions.

The subject (patient) can be tested prior to injury to gather normative cognitive and motor function data for each subject and also immediately after an injury and during a recovery process. This can be accomplished by the following steps.
1. The subject arrives at stationary or portable virtual reality (VR) laboratory (lab) location.
2. The subject's demographic and historical health information can be entered into the CPU and software.
3. The testing process can be described to subject.
4. The subject can be placed into safety harness in front of a virtual reality screen or display.
5. The virtual reality software modules can be operated or run, one at a time, by a clinician, such as a physician, nurse or medical technician. The clinician can use the master menu to select each module to be run.
6. The modules can present the subject with multiple virtual environments and capture data regarding their experience within these environments.
7. The reporting module can analyze data and score the subject's cognitive and motor function and identify specific deficiencies.
8. At the clinician's or doctor's recommendation, the rehabilitation modules can be used to address the areas of cognitive and motor deficiency.
9. During the course of any rehabilitative programs, the assessment modules can be used to track the subject's progress.

The inventive and process can use three (3) software modules to assess working memory, also known as short term memory. These modules can be used to test two different types of working memory: spatial navigation and object recall/recognition.

The Virtual Hospital Corridor (VHC) software module addresses the assessment of spatial memory impairment in the context of interactions with an everyday environment without sacrificing analytic control. Spatial memory can be defined as one's understanding and recall of their current physical environment and their orientation within that environment. A normally functioning spatial memory allows the subject the capability of creating a cognitive map for navigation in both new and previously experienced spatial environments. Traumatic brain injury (TBI) and other cognitive and motor function deficiencies can seriously disrupt this ability.

Object recognition impairment can also be addressed within the Virtual Hospital Corridor (VHC) software module. A subject's ability to recognize and recall visual clues (i.e. objects common to the environmental setting) in conjunction with navigational tasks can be impaired by TBI and other cognitive and motor function disorders.

The Virtual Hospital Corridor (VHC) environment can be based on a series of real hospital corridors, comprising a pathway or hallway with a number of common hospital objects along the way. The subject (patient or TBI person) can be required to navigate through the virtual corridor using a 3D mouse or other interactive devices to reach a specified destination, or the subject may be passively taken through the virtual corridor to view the targeted pathway and/or objects. As the subject moves through the virtual environment (VE), the subject may need to remember certain hospital objects seen along the pathway. The subject can use these common hospital objects seen along the way as navigational clues. Various degrees of complexity are possible within the VHC software module in order to properly assess diminished memory processes (i.e., encoding, retention, retrieval) in subjects suffering from mild-to-severe TBI and other cognitive motor function deficiencies. The varying degrees of complexity can be based on the parameters such as the number of turns, different pathways, different destination points, speed of navigation and quantity of pathway objects and can be controlled by the clinician. Lists of the items to be recognized, recalled, displaced, and/or arranged in specific order are pre-programmed (e.g., visual objects, pictures, paths, directions, temporal sequence of observed events, objects' spatial location, etc.) by the clinical users via the specially developed interactive software.

The Spatial Navigation Module 1 tests the subject's spatial memory within the Virtual Hospital Corridor with a task of navigating from a starting point to a destination and then returning to the starting point. In an example of the process for using the Spatial Navigation 1 assessment module:

1. The subject is currently located in Virtual Hospital Room (e.g. Room #1001).

2. The subject must visit the bathroom down the hall and return back to Room #1001.

3. The correct pathway to the bathroom and back to Room #1001 will be shown via a passive demo animation.

4. The subject's task is to remember the correct path and use it to actively navigate to the bathroom and then back to Room #1001.

5. The subject can actively navigate the virtual hallways using an interactive device.

6. The subject is instructed to move through the hallways as fast as possible without making any errors.

7. Any errors in navigation will result in the end of the exercise. The passive demo animation of the correct pathway can then be shown again.

8. The subject will have three trials to correctly accomplish the navigation task.

9. The degree of the subject's navigational success can be captured and scored in the reporting module.

The Spatial Navigation Module 2 tests the subject's spatial memory within the Virtual Hospital Corridor with a task of navigating from a starting point to a destination. The degree of navigational complexity can be varied by the clinician. In an example of the process for using the Spatial Memory 2 assessment module:

1. The subject is currently located at a starting point in the corridor.

2. The subject must navigate from the starting point to a target location in the virtual hospital.

3. The correct pathway to the target location can be shown to the subject via a passive demo animation.

4. The subject's task is to remember the correct path and use it to actively navigate to the target location.

5. The subject can navigate the virtual hallways using an interactive device.

6. The subject can be instructed to move through the hallways as fast as possible without making any errors.

7. The clinician can vary the level of difficulty of the correct pathway to the target location.

8. The degree of the subject's navigational success can be captured and scored in the reporting module.

The Object Recognition Modules (A & B) can focus on the various common hospital objects the subject passes during a journey through the Virtual Hospital Corridor (VHC). In an example of Part A of the process of using the Object Recognition Module in the novel process and system, the subject (patient) can be passively moved ('auto-piloted') through the corridors and can see seven (7) various objects along the way, one at a time. The subject can then be instructed to select, from a larger group of objects, only the seven (7) objects seen along the path.

1. The subject can be passively taken on a journey through the virtual hospital corridors.

2. Along the way, the subject can see seven (7) common hospital objects such as a walker, wheelchair, intravenous (IV) stand, couch, hospital bed, etc.

3. The subject must remember these seven (7) virtual objects.

4. At the end of the passive journey through the hallways, the virtual environment can display an interactive panel with a visual list of fourteen (14) objects, including the seven (7) that were seen during the passive journey.

5. The subject's task is to recall and, using the joystick or other interactive control device, to select only the seven (7) objects that were seen during the passive journey through the virtual hospital corridors.

6. The subject's score of correct selections will be captured and scored in the reporting module.

In an example of Part B of the Object Recognition Module, the virtual environment generated by the novel process and system can be displayed to the subject (patient) as a virtual panel with a visual list of seven (7) common hospital objects. The subject can be asked to memorize these objects. The subject can then be passively moved or auto-piloted through the virtual corridors and can see multiple objects along the way, one at a time. As the virtual objects are seen, the subject must select only those previously presented in the visual list.

1. The novel process and system can display a virtual panel with seven (7) common hospital images to the subject via the interactive panel.

2. The subject can have 60 seconds to commit these images to memory.

3. The object recognition module and software can then take the subject on a tour of the virtual hospital. Along the journey, multiple objects can be seen, one at a time.

4. As the subject recognizes a previously shown object, they must use the Interactive Device to select the object.

5. The subject should only select the seven (7) objects presented earlier.

6. The subject's score of correct selections can be captured and scored in the Reporting Module.

The assessment of attention deficits in traumatic brain injury (TBI), such as deficits in visual selective and sustained attention, is a prominent aspect of cognitive dysfunction after TBI. Other cognitive and motor function deficits, such as parietal brain cortex lesions, also have the potential to negatively impact subjects' attention. Subjects (patients) with attention deficit disorders frequently complain of distractibility and difficulty attending to more than one thing or task at a time. The Virtual Elevator software module focuses on subject attention measurement utilizing a virtual reality based test of everyday attention (TEA) within the context of a dual-task paradigm.

In an example of the Virtual Elevator of the Attention Module of the process and system:

1. The subject is placed in front of the Virtual Elevator image.

2. The novel process and system with the Attention Module and software can move the virtual image to give the subject (patient) the sense that the elevator is either rising or descending between floors numbered 1 through 12. The virtual elevator can move up to any floor from 1 to 12 and down to any floor 12 to 1. There are visual separations between the floors that the subject can see and count in order to identify the current floor upon arrival (stop).

3. At the start of each trial (test), the current floor number is identified to the subject within the image. The virtual elevator then closes its virtual doors and begins to move up or down. As the virtual elevator moves, the subject will see the floors pass but no floor numbers are indicated. The subject must count each floor as it passes. When the elevator stops, the subject must identify the correct floor number by pressing the corresponding number on the elevator control panel. The virtual elevator doors will then open and the virtual wall at the far end of the virtual visible corridor will display the current floor number to the subject.

4. There are numerous random trials that can last for up to 10 minutes and the number of correct and/or incorrect floor identification responses are captured and stored in an output file.

5. Elevator floor counting with distractions where additional sources of noise with external visual and audio stimuli (e.g., adjacent buildings, windows, trees, people coming in and out, different sounds on each floor, etc.) can be added to vary the degree of complexity of the task as a function of subjects' current status.

The CPU and software then analyzes the data to determine the subject's degree of Sustained and Selective Attention.

The Virtual Hospital Room (VHR) balance software module concentrates on Balance assessment within the scope of visual-kinesthetic integration. Deficits and abnormalities in balance and postural control due to traumatic brain injury (TBI) and other cognitive and motor function deficiencies can often pass undetected in standard post-injury cognitive and physical tests. The CPU and VHR balance software module allows for a unique method of detection for these symptoms through the assessment of the subject's visual motor integration.

In an example of the process of using the Virtual Hospital Room balance module in the novel process and system:

1. The subject is placed in front of the stationary virtual hospital room (VHR) image while standing on a force platform. When the VHR is stationary, the subject is asked to remain as stable as possible with their feet flat on the force platform, hands at their side and eyes looking straight ahead. If the subject is unable to stand, they may perform this task in a seated position using a modified force platform system.

2. The CPU and software of the novel process and system can then begin to move the VHR on the X, Y and/or Z axes.

3. The VHR may appear to be shifting to the left, right, forwards or backwards for various intervals, such as 30 second intervals. As the projected image of the VHR moves, pans and shifts, the subject (patient) will naturally respond to the image's movement by shifting his/her whole body. The natural response from a healthy subject is to sway with the same amplitude and frequency as the virtual image on the screen. The TBI subject and those with other neurocognitive deficiencies are not able to follow the movement or may get dizzy or sick, which is a natural response for their condition.

4. During the trial (test), the subject can stand on a force plate that measures and captures data on each movement the subject makes in response to the shifting image.

5. This data is then analyzed and scored by the reporting module with the CPU to determine the subject's degree of visual motor integration.

The Virtual Body (VB) module or praxis Body Awareness module assesses the patient's spatial awareness by presenting the TBI patient with a series of 3D body images that, when arranged in the correct order illustrate the proper physical actions that need to be executed to complete a task such as moving from a seated position to a standing position or vice versa or stepping over an obstacle. TBI and other cognitive and motor function deficiencies can cause the patient to have difficulty correlating body movements with the physical actions they desire to complete.

Various degrees of complexity in spatial sequencing designs assess praxis impairment at various stages of TBI and cognitive dysfunction. There are three dimensions of spatial abilities: (1) spatial relations and orientation (e.g. associate the word “shoulder” with an image of the appropriate body part); (2) visualization, which is the ability to create and recreate past and future experiences; and (3) kinesthetic imagery, which is the ability to determine the spatial position of an object in relation to oneself.

Each of these spatial abilities are selectively sensitive towards traumatic brain injury (TBI) and cognitive dysfunction resulting in a wide spectrum of higher-order motor disorders affecting the performance of skilled, learned movement. Specifically, deficits in the conceptual system (ideational apraxia) or in production (ideomotor apraxia) may result in the subject's inability to imitate proper postures and complex actions requiring the use of various objects movement (e.g. chairs during sitting/standing tasks) due to abnormal integration between two systems (i.e., praxis deficit). An example of a virtual reality (VR)-based praxis test implemented via spatial sequencing virtual body module is shown in FIGS. 17 and 18.

In an example of the process of using the body awareness module in the novel process and system, the patient's task is to rotate 3D virtual body images comprising a realistic looking virtual person, from in face to profile views and then thereafter to correctly arrange these 3D virtual images from a random to an organized manner (e.g., from sitting to standing postures) according to instructions via human-computer interface using an interactive device (e.g. 3D mouse).

In the example of the process of using the body awareness module in the novel process and system, the following steps can occur:

1. The subject (patient) is placed in front of the virtual environment generated by the CPU.

2. The Virtual Body software module and CPU can display a randomly arranged sequence of five (5) 3D virtual body posture images that collectively illustrate a body action such as moving from the sitting position to the standing position or vice versa or stepping over an obstacle.

3. Using an interactive device, the subject should first highlight and rotate each image from the in-face view (front-on) to the side-view (profile) to fully view the body image.

4. After fully viewing each body image, the subject should use the interactive device to electronically drag each image into the correct sequential order to accurately illustrate the body action (e.g. a sitting position to a standing position).

5. The subject should accomplish these tasks as fast as possible without error in positioning the images in the correct sequence.

6. The Body Awareness software module can record the subject's success rate, time of completion, etc and then analyze, score and display the results in the reporting module.

The CPU and software modules generate and capture data based on the subject's performance of the specific given tasks. This data is placed in data storage directories for access by the Reporting Module. The CPU in conjunction with the Reporting Module can analyze and score the subject's testing results. Independent module scores can be reported. Independent module scores can also combined and an overall cognitive motor function score can be determined and reported. Independent and combined scores can also be compared to a testing results database. The Reporting Module can display and generate a report comprising a severity index for the TBI or other cognitive and motor function deficiency. This is a relative rating scale that can be used to compare a subject's performance over time. The subject's performance can also be compared to the subject's prior performances or to other subjects' results. Clinicians can use this scale to determine appropriate timing for a subject's return to normal activity, e.g. an athlete's return to play or a soldier's return to duty

The procedure for using the reporting module in the novel process and system can include the following steps:

1. The clinician (doctor, nurse or medical technician) will use the reporting menu to select the report(s) that will be output by the reporting module.

2. The clinician will be able to select if subject's personal information is included or omitted from the results report.

3. As each software module subject trial is run, the data can be generated, captured, analyzed and prepared for a results report.

4. At the end of each software trial, the reporting module will generate a non-editable results report for each independent software module.

5. The results report can also include a severity index for the TBI, deficient cognitive function or deficient motor function that allows a comprehensive view of the combined software module outcomes.

6. The clinician can save, display, print and e-mail the results report.

The CPU and software modules use dynamic build processes to construct each set of basic elements within a virtual environment. The flowcharts illustrate the process (method) of creating, integrating and controlling the following:

1. Virtual Environment (VE)—the interactive and immersive virtual world that the subject can see on the screen during assessment and rehabilitation (FIG. 20).

2. Visual Stimuli—the image displaying the current state of VE on the screen which a subject can see on the screen (FIG. 20).

3. Walls—e.g. six (6) rectangular planes which define the spatial boundaries of a BLOCK (FIG. 20); The six planes that comprise the WALLS and a BLOCK are: (a) left wall; (b) right wall; (c) top, sky or ceiling; (d) bottom, floor or ground; (e) front or far wall; and (f) rear or back wall.

4. Props—an object, furniture, buttons and other pieces of geometry that the subject views and sometimes interacts with while in the VE (FIG. 20).

5. VE Geometry—collection of all visible objects representing WALLS and PROPS.

6. VE Perturbation—visual movement of the VE according to a defined motion pattern (e.g. sinusoidal oscillation, abrupt translation or rotations, etc.).

7. Path—a sequence of VE BLOCKS which the subject should navigate through while moving from a starting position to a target destination.

8. Path Segments—a sequence of VE BLOCKS which the subject should navigate through while moving from a starting position to a target destination. A Path may be comprised of multiple Path Segments.

9. Interactive Devices—the various pieces of external hardware (e.g. joystick, force plate, etc) that collect subjects' response data within the virtual environment and/or allow the subjects to interact and control the virtual environment.

10. Panel or Interactive Panel—A screen presented to the subject within the VE that allows the subject to view objects, make selections or further interact with the application.

11. Data Structure or Struct—a complex variable in C and C++ that can hold a group of variables (FIG. 36).

12. List or Linked List—implemented by linking each Data Structure; has a link to the previous Data Structure as well as the next Data Structure (FIG. 38); and

13. Completed List of BLOCKS (or WALLS, PROPS, PANEL) Data Structures—a complex variable holding a set of linked Data Structures of certain type (FIG. 37).

As an example, within the memory modules, a set of equally spaced PROPS positioned in rows and columns can be presented to the subject in a certain area of the VE interactive panel where any PROP can be selected by the subject using Interactive Devices (FIG. 10).

The audible sounds and speech that can be used in and with the modules are primarily used as distractions to increase the complexity of tasks. The software module can ask the subject (patient) a set of simple questions to distract them while they are completing tasks (e.g. What sports do you play?, What is your hometown?).

Sounds such as construction noises, music, birds singing, crowds cheering, etc can be used with certain modules to leverage the subjects' sense of hearing and increase complexity or to provide audio clues to specific locations. When in the Virtual Elevator, the subject can hear construction sounds when passing a certain floor each time. The clinician can test the subject's association of sound with location.

The inventive system, process and software can function with virtual reality (VR) hardware projection and display systems and interactive devices. Additional technology can be leveraged in conjunction with the software.

Electroencephalograph (EEG) can be recorded from the subject during the sessions of the novel process and system to provide brain-imaging feedback.

The inventive system, process and software can work within a Functional Magnetic Resonance Imaging (fMRI) machine. The fMRI can provide brain scans of subjects while the HeadRehab software is in use.

After using the assessment software modules, the Reporting Module and CPU can generate an assessment of the various cognitive and motor functions tested for the subject (patient) as well as a relative scale rating of the subject's overall cognitive and motor function. The clinician, doctor or researcher can then decide what means of rehabilitation, if any, the subject should undertake. The rehabilitation software modules can be used for treatment of identified areas of cognitive motor dysfunction.

An assessment results database can be created. This can occur once subject consent is gained and the data collected need not contain any personally identifiable subject data. Demographics (gender, age, fitness level, etc) and assessment data and scores can be compiled to enhance the relative database used for subject results evaluation.

Among the many advantages of the novel process and system for assessment and rehabilitation of cognitive and motor functions using virtual reality, are:
1. Superior process and system.
2. Outstanding performance.
3. Higher sensitivity.
4. Superb results.
5. Excellent assessments.
6. Better diagnosis.
7. User-friendly.
8. Reliable.
9. Helpful for doctors, nurses, medical technicians and other clinicians.
10. Safe.
11. Portable.
12. Comfortable to user.
13. Easy to use.
14. Economical.
15. Faster diagnosis, assessment and rehabilitation.
16. Efficient.
17. Effective.
18. Prevents cheating.
19. Minimizes and eliminates undesirable learning effects.
20. Helps assure objective results.
21. Cognitive and motor tests are presented in a safe, completely controllable laboratory setting.
22. Immersive 3D virtual environment can create sense of presence for the subject (patient).
23. Sense of presence in virtual environment can generate true-to-life, realistic responses that enhance the quality of the test results.
24. Tasks and environments can be transferable to real-life situations that are familiar to the subjects (patients).
25. Virtual environment can be adapted to any real-life environment to enhance subject familiarity and/or meet researcher requirements (e.g. hospital settings, sports environments, military surroundings, school or university locations).
26. Dynamic software, module and process design allows for varying levels of complexity and module expansion.
27. Standardized, proprietary scoring system allows for subject results comparison: against normative (normal) baseline data for that specific subject (patient) over time to determine rehabilitation progress as well as between different subjects and against collected database for pre and post injury or neurocognitive condition onset.
28. Complexity and organization of tests that are adjustable by clinicians.
29. Assessment software and rehabilitation software modules can be used separately or together.
30. Assessment software modules can be used independently to target specific cognitive function areas (e.g. memory, attention) and motor function areas (i.e. balance) or in combination to present a broad cognitive and motor function evaluation.
31. Rehabilitation software modules can also be used independently to target specific cognitive areas (e.g. memory, attention) and motor function areas (e.g. balance) or in combination to rehabilitate multiple cognitive and motor functions.
32. Assessment software modules can be utilized as a progress measurement tool for cognitive and/or motor function rehabilitative programs.
33. Software modules can be used with a portable virtual reality system or in a stationary virtual reality lab.
34. Software modules, CPU and the novel system and process do not require advanced medical degree or special advanced technical training to operate.
35. Software can be deployed on Unix, Linux or Windows platforms.
36. Software modules and hardware as well as the novel process and system can be adapted to meet requirements of special-needs subjects (patients).
37. Flexible hardware options can allow for low cost of entry into virtual reality environments and systems.
38. Cognitive and motor assessment may be incorporated into annual physical examination to monitor cognitive and motor functions of the subject throughout lifetime to capture result deviations from preceding tests, which may be due to TBI or other neurological disorders related to aging or injuries.

Although embodiments and examples of the invention have been shown and described, it is to be understood that various modifications, substitutions and rearrangements of modules, parts, components, equipment and/or process (method) steps, as well as other uses of the novel process and system, can be made by those skilled in the art without departing from the novel spirit and scope of this invention.

What is claimed is:

1. A process for use with a person having a traumatic brain injury or a neurocognitive disorder, comprising:
generating a virtual reality environment (VRE) comprising at least one image with a central processing unit (CPU;
electronically displaying the VRE to a person having a traumatic brain injury (TBI) or other neurocognitive disorder;
identifying a task to be performed by the person in the VRE;
performing the task by the person;
electronically imputing interactive communications comprising the person's performance of the task to the CPU with an electronic interactive communications device;
electronically evaluating the person's performance in the CPU based on the electronically inputted interactive communications to the CPU; and
electronically assessing the person's impairment and extent of the TBI or other neurocognitive disorder by electronically determining a deficiency of at least one of the person's functions selected from the group consisting of a cognitive function and a motor function, as a result of the TBI or other neurocognitive disorder.

2. A process in accordance with claim 1 wherein;
said VRE is a three-dimensional (3D) virtual reality environment; and
said images are moveable.

3. A process in accordance with claim 1 wherein said task is performed by the person via the interactive communications device.

4. A process in accordance with claim 1 wherein:
said cognitive function is selected from the group consisting of memory, recall, recognition, attention, spatial awareness, body awareness and combinations thereof; and
said motor function comprises balance.

5. A process in accordance with claim 1 wherein the task is selected from the group consisting of: object recognition, virtual navigation, virtual walking, virtual steering, spatial navigation, object navigation, spatial memory, kinesthetic imagery, virtual arrangement of images, standing, balance and memorizing objects.

6. A process in accordance with claim 1 wherein the image comprises a virtual image selected from the group consisting of: a virtual elevator, virtual elevator buttons, virtual hospital corridor, visual body, virtual hospital room, virtual bathroom, virtual door, virtual hall, virtual wall, virtual room, virtual pathway, virtual object, virtual sign, virtual picture, virtual bed, virtual cart, virtual stretcher, virtual person, virtual table, virtual positions of a person's body, virtual furniture, virtual walker, virtual wheelchair, virtual hospital bed, virtual couch, and virtual stand.

7. A process in accordance with claim 1 wherein said VRE comprise multiple virtual environments with visual distractions and/or audible distractions for the person performing the task.

8. A process in accordance with claim 1 including:
electronically inputting and recording performance data in the CPU; and
electronically reporting the performance data from the CPU.

9. A process in accordance with claim 8 including:
electronically comparing the performance data with the person's prior performance data or normal performance data from a data base; and
electronically outputting or printing the comparison data.

10. A process in accordance with claim 9 including:
electronically scoring the comparison data in the CPU;
electronically reporting the score from the CPU; and
using the score to help rehabilitate the person having TBI.

11. A process for use with a person having a traumatic brain injury, comprising;
generating a three-dimensional (3D) virtual reality environment (VRE) comprising at least one scene with moveable virtual images with a central processing unit (CPU) in conjunction with at least one module;
electronically displaying the VRE on a screen to a person having a traumatic brain injury (TBI);
identifying a task to be performed by the person in the VRE;
electronically performing the task by the person with an electronic interactive communications device;
electronically inputting interactive communications comprising the person's performance of the task to the CPU with the electronic interactive communications device;
electronically evaluating the person's performance in the CPU based on the electronically inputted interactive communications to the CPU;
electronically assessing the person's impairment and extent of TBI or other neurocognitive disorder by electronically determining a deficiency of at least one of the person's functions selected from the group consisting of a cognitive function and a motor function;
said cognitive function is selected from the group consisting of memory, recall, recognition, attention, spatial awareness, body awareness and combinations thereof; and
said motor function comprises balance.

12. A process in accordance with claim 11 wherein:
said CPU is selected from the group consisting of a computer, computer workstation, laptop, desktop computer, portable computer, microprocessor, computer system, iPad, tablet computer, wireless computer, wired computer, netbook, electronic communications device, portable networking device, internet communication device, mobile phone, flip phone, camera phone, clamshell phone, radio telephone, cellular phone, smart phone, tablet phone, portable media payer (PMP), personal digital assistant (PDA), wireless e-mail device, handheld electronic device, mobile electronic device, video game device, video game console, video game player, electronic amusement device for use with a television or monitor, video gaming device, and combinations of any of the preceding;
said interactive communications device is selected from the group consisting of an electronic joystick, electronic mouse, three-dimensional (3D) electronic mouse, electronic controller, navigation controller, handheld controller, fMRI-compatible mouse, wireless controller, wired controller, voice activated controller, video game controller, inputting device, key pad, screen pad, touch pad, keyboard, treadmill, motion tracking device, force platform, force plate, wireless interactive communications and combinations of any of the preceding; and
said screen is selected from the group consisting of a portable screen, touch screen, computer screen, touch pad, display, monitor, wall, shade, liquid crystal screen, projection screen, video projection screen, video screen, television, high definition television, 3D television, virtual reality goggles and virtual reality headset.

13. A process is accordance with claim 11 wherein:
said module comprises at least one module selected from the group consisting of a memory module, spatial memory module, recognition module, object recognition module, attention module, body module, body awareness module, and results reporting module;
task is selected from the group consisting of: object recognition, virtual navigation, virtual walking, virtual steering, spatial navigation, object navigation, spatial memory, kinesthetic imagery, virtual arrangement of images, standing, balance and memorizing objects; and
the virtual images are selected from the group consisting of a virtual elevator, virtual elevator buttons, virtual corridor, virtual hospital corridor, visual body, virtual hospital room, virtual bathroom, virtual door, virtual hall, virtual wall, virtual room, virtual pathway, virtual object, virtual sign, virtual picture, virtual bed, virtual floor, virtual cart, virtual stretcher, virtual person, virtual table, virtual positions of a person's body, virtual furniture, virtual walker, virtual wheelchair, virtual hospital bed, virtual couch, virtual stand and combinations of the preceding

14. A process in accordance with claim 11 wherein said VRE comprise multiple virtual environments with visual distractions and/or audible distractions for the person performing the task.

15. A process in accordance with claim 11 including:
electronically inputting and recording performance data in the CPU;
electronically reporting the performance data from the CPU.
electronically comparing the performance data with the person's prior performance data or normal performance data from a data base;
electronically outputting or printing the comparison data;
electronically scoring the comparison data in the CPU;
electronically reporting the score from the CPU; and
using the score to help rehabilitate the person having the TBI or other neurocognitive disorder.

16. A process for use with a person having a traumatic brain injury or other neurocognitive disorder, comprising;
generating a three-dimensional (3D) virtual reality environment (VRE) comprising at least one scene with moveable virtual images with a central processing unit (CPU) in conjunction with at least one software module;
electronically displaying the VRE on a screen to a person with a traumatic brain injury (TBI) or other neurocognitive disorder;
identifying a task to be performed by the person in the VRE;
the person with the TBI electronically performing the task with an electronic interactive communications device;
electronically inputting interactive communications to the CPU with the electronic interactive communications device, the interactive communications comprising the person's responses and performance of the task;
electronically evaluating the person's performance in the CPU based on the electronically inputted interactive communications to the CPU;
electronically assessing the person's impairment and extent of TBI or other neurocognitive disorder in the CPU by electronically determining a deficiency if at least one of the person's functions selected from the group consisting of a cognitive function and a motor function; the cognitive function being selected from the group consisting of memory, recall, recognition, attention, spatial awareness, body awareness and combinations thereof; the motor function comprising balance;
the CPU being selected from the group consisting of a computer, computer workstation, laptop, desktop computer, portable computer, microprocessor, computer system, ipad, tablet computer, wireless computer, wired computer, netbook, electronic communications device, portable networking device, internet communication device, mobile phone, flip phone, camera phone, clamshell phone, radio telephone, cellular phone, smart phone, tablet phone, portable media payer (PMP), personal digital assistant (PDA), wireless e-mail device, handheld electronic device, mobile electronic device, video game device, video game console, video game player, electronic amusement device for use with a television or monitor, video gaming device, and combinations of any of the preceding;
the interactive communications device being selected from the group consisting of an electronic joystick, electronic mouse, three-dimensional (3D) electronic mouse, electronic controller, navigation controller, handheld controller, fMRI-compatible mouse, wireless controller, wired controller, voice activated controller, video game controller, inputting device, key pad, screen pad, touch pad, keyboard, treadmill, motion tracking device, force sensing device; force platform, force plate, wireless interactive communications and combinations of any of the preceding;
the screen being selected from the group consisting of a portable screen, touch screen, computer screen, touch pad, display, monitor, wall, shade, liquid crystal screen, projection screen, video projection screen, video screen, television, high definition television, 3D television, virtual reality goggles and virtual reality headset;
the module comprising at least one module selected from the group consisting of a memory module, spatial memory module, recognition module, object recognition module, attention module, body module; body awareness module, and results reporting module;
the task being selected from the group consisting of: object recognition, virtual navigation, virtual walking, virtual steering, spatial navigation, object navigation, spatial memory, kinesthetic imagery, virtual arrangement of images, standing, balance, and memorizing virtual objects;
the virtual images comprising virtual 3D images selected from the group consisting of a virtual elevator, virtual elevator buttons, virtual corridor, virtual hospital corridor, visual body, virtual hospital room, virtual bathroom, virtual door, virtual hall, virtual wall, virtual room, virtual pathway, virtual object, virtual sign, virtual picture, virtual bed, virtual floor, virtual cart, virtual stretcher, virtual person, virtual table, virtual positions of a person's body, virtual furniture, virtual walker, virtual wheelchair, virtual hospital bed, virtual couch, virtual stand and combinations of the preceding;
electronically reporting the performance data from the CPU;
electronically comparing the performance data with the person's prior performance data or normal performance data from a data base;
electronically scoring the comparison data in the CPU;
electronically reporting the score and comparison data from the CPU; and
using the score to help rehabilitate the person with the TBI or other neurocognitive disorder.

17. A process in accordance with claim 16 wherein:
a safety harness is placed on the person with the TBI or other neurocognitive disorder before the person performs the task; and
the electronic reporting is selected from the group consisting of electronically displaying the score and comparison data from the CPU on the screen, printing the score and comparison data from the CPU in a printed report; and
combinations thereof.

18. A process in accordance with claim 17 wherein:
the task is selected from the group consisting of virtual navigation, virtual walking, spatial navigation, and combinations thereof;
the task is electronically performed by virtually arriving at the virtual destination;
the module is selected from the group consisting of a memory module, spatial memory module, recognition module, object recognition module, and combinations thereof;
the virtual 3D images are selected from the group consisting of a virtual corridor, virtual hospital corridor, virtual hospital room, virtual room, virtual pathway, virtual floor, virtual bed, virtual bathroom, and combinations thereof;

19. A process in accordance with claim 18 wherein:
the module further includes at least one module selected from the group consisting of object recognition module and body awareness module;
electronically performing the task includes
identifying virtual 3D objects in the virtual hospital corridor in cooperation with the object recognition module; or
identifying the order of virtual body positions in cooperation with the body awareness module to help the person with the TBI to sit and stand.

20. A process in accordance with claim 18 wherein:
the module further comprises a balance module and an attention module;
the interactive communications device includes a force sensing device selected from the group consisting of a force platform and force plate;
the task includes standing and balancing on the force sensing device in cooperation with the balance module;
the virtual 3D images comprise a virtual elevator, virtual door, and virtual floors for use with the attention module;
the task is performed by recognizing floor numbers in response to virtual movement of the virtual elevator; and
the VRE comprise multiple virtual environments with distraction for the person with TBI performing the task; and
the distractions comprise electronic distractions selected from the group consisting of electronic visual distractions on the screen and electronic audible distractions.
